(19) United States
(12) Patent Application Publication
(10) Pub. No.: US 2023/0056493 A1
(43) Pub. Date: Feb. 23, 2023

(54) MEDICAL POLE, COMPONENTS, AND IDENTIFICATION SYSTEM

(71) Applicant: Fisher & Paykel Healthcare Limited, Auckland (NZ)

(72) Inventor: Martin Paul Friedrich KRAMER, Auckland (NZ)

(21) Appl. No.: 17/629,276

(22) PCT Filed: Jul. 23, 2020
(86) PCT No.: PCT/IB2020/056933

§ 371 Date: Jan. 21, 2022

Related U.S. Application Data
(60) Provisional application No. 62/878,478, filed on Jul. 25, 2019.

(51) Int. Cl. B60B 33/02; B62B 9/08

(52) U.S. Cl. CPC B60B 33/02; B62B 9/087

(57) ABSTRACT

A medical pole 1 has a movable base 103 supporting a pole member 3. The movable base 103 a frame 105 and two or more wheels. A first wheel 111 has a first mode in which the first wheel 111 is swivelable about a respective upright axis relative to the frame and is rollable about a respective transverse axis, and a second mode in which the first wheel 111 is rollable about the respective transverse axis but is non-swivelable about the respective upright axis. The first wheel 111 is configurable between the first mode and the second mode.

### TECHNICAL FIELD

The present disclosure relates to a medical pole to support one or more medical accessories in a medical environment. The present disclosure also relates to components of a medical pole. The present disclosure also relates to an identification system for a medical pole or other medical apparatus.

### BACKGROUND ART

Mobile medical poles (also known by other names such as hospital poles, IV poles, rolling carts, mobile infusion stands, medical pole stands) comprising a long upwardly extending pole member typically mounted on a wheeled base are used in medical environments such as a patient care facility, hospital, surgery, or home care environment. Generally, such medical poles are used for carrying medical equipment (e.g. a breathing apparatus) and/or medications (e.g. intravenous medication). The medical poles may be portable between different locations due to plurality of wheels/castors mounted at the bottom.

### SUMMARY

It would be desirable to provide a medical pole or component(s) for use therewith that provides ease of manoeuvring and control of a medical pole.

In accordance with certain features, aspects and advantages of at least one of the embodiments disclosed herein, a medical pole is disclosed, the medical pole comprising: a movable base supporting a pole member, the movable base comprising a frame and two or more wheels, the two or more wheels comprising a first wheel having a first mode in which the first wheel is swivelable about a respective upright axis relative to the frame and is rollable about a respective transverse axis, and a second mode in which the first wheel is rollable about the respective transverse axis but is non-swivelable about the respective upright axis, wherein the first wheel is configurable between the first mode and the second mode.

In some configurations, the two or more wheels comprise a second wheel having a first mode in which the second wheel is swivelable about a respective upright axis relative to the frame and is rollable about a respective transverse axis, and a second mode in which the second wheel is non-rollable about the respective transverse axis and is non-swivelable about the respective upright axis, wherein the second wheel is configurable between the first mode and the second mode.

In some configurations, the first wheel and the second wheel are arranged substantially opposite each other, with the pole member located substantially between the first wheel and the second wheel.

In some configurations, the first wheel and/or the second wheel has a locking mechanism to enable a user to select the two modes.

In some configurations, the first wheel has a fixed orientation about the respective upright axis relative to the frame in the second configuration mode.

In some configurations, the first wheel has one or more fixed orientation positions about the respective upright axis.

In some configurations, the first wheel has four fixed orientation positions about the respective upright axis.

In some configurations, the fixed orientation positions are substantially orthogonal to each other.

In some configurations, the first wheel is able to move into the one or more fixed orientation positions if the first wheel is not in such position when the locking mechanism of the first wheel is activated.

In some configurations, at least one of the fixed orientation positions is parallel to an intended movement direction of the medical pole.

In some configurations, the first wheel has a third mode in which the first wheel is non-rollable about the respective transverse axis and is non-swivelable about the respective upright axis, wherein the first wheel is configurable between the first mode, the second mode, and the third mode.

In some configurations, the orientation of the second wheel about the respective upright axis relative to the frame is fixed upon activation of the locking mechanism of the second wheel.

In some configurations, the movable base comprises a third wheel.

In some configurations, the third wheel is located generally between and offset from the first wheel and the second wheel.

In some configurations, the third wheel has a first mode in which the third wheel is swivelable about a respective upright axis relative to the frame and is rollable about a respective transverse axis, and a second mode in which the third wheel is non-rollable about the respective transverse axis and is non-swivelable about the respective upright axis, wherein the third wheel is configurable between the first mode and the second mode.

In some configurations, the movable base comprises a fourth wheel.

In some configurations, the fourth wheel is located generally between and offset from the first wheel and the second wheel, generally between and offset from the second wheel and the third wheel, or generally between and offset from the third wheel and the first wheel.

In some configurations, the fourth wheel located substantially on an opposite side of the base to the third wheel.

In some configurations, the fourth wheel has a single mode in which the fourth wheel is swivelable about a respective upright axis relative to the frame and is rollable about a respective transverse axis.

In some configurations, the fourth wheel has a first mode in which the fourth wheel is swivelable about a respective upright axis relative to the frame and is rollable about a respective transverse axis, and a second mode in which the fourth wheel is non-rollable about the respective transverse axis and is non-swivelable about the respective upright axis, wherein the fourth wheel is configurable between the first mode and the second mode.

In some configurations, the frame comprises at least one extension leg with a respective one of the wheels arranged along the extension leg.

In some configurations, the frame comprises a plurality of the extension legs, each having a respective one of the wheels arranged along the extension leg.

In some configurations, the extension leg(s) extend(s) perpendicularly relative to the pole member.

In some configurations, the pole member comprises one or more retaining features for retaining a medical device or a medical device component.

In some configurations, the medical pole comprises a handle extending from the pole member.

In some configurations, the handle extends from the pole member in a direction that is substantially perpendicular to an axis extending through the first wheel and the second wheel.

In some configurations, the handle extends from the pole member in a direction away from the first wheel.

In some configurations, the handle extends from the pole member in a direction away from the first wheel that is transverse or orthogonal to the first wheel or extends from the pole member in a direction that is opposite to the first wheel

In some configurations, the handle extends from the pole member in a direction that is substantially parallel to and over an extension leg.

In some configurations, the first wheel and/or the second wheel and/or the third wheel has a locking mechanism to enable a user to select the two modes, wherein the locking mechanism comprises a depressible lever.

In some configurations, the locking mechanism comprises a tooth/teeth and slot(s) arrangement, wherein the lever is configured to actuate the tooth/teeth and slot(s) toward one another for engagement to lock swiveling of the wheel.

In some configurations, the first wheel comprises fewer teeth and slots compared to the second wheel and/or the third wheel.

In some configurations, at least one locking mechanism comprises a brake member that is actuable by the lever to engage a surface of the wheel that contacts a ground surface when in use.

In accordance with certain features, aspects and advantages of at least one of the embodiments disclosed herein, a medical pole handle is disclosed, the medical pole handle comprising: a handle body with a grip end portion and a mounting end portion; a mount that couples the handle to a pole member of a medical pole that has a longitudinal axis; and a fastening mechanism to couple the handle body with the mount in two or more orientations with respect to the mount, wherein in one of the orientations the grip end portion extends in a direction that is transverse to the pole member and generally horizontal relative to a ground surface, and is non-intersecting with the longitudinal axis of the pole member.

In some configurations, the orientations are about a longitudinal axis of handle body.

In some configurations, the handle body is orientable in at least two planes.

In some configurations, the two planes are transverse to each other.

In some configurations, the handle body comprises an intermediate portion between the grip end portion and the mounting end portion, wherein the longitudinal axis extends through a portion of the intermediate portion.

In some configurations, the grip end portion is transverse to the intermediate portion.

In some configurations, an end of the intermediate portion is connected to an end of the grip end portion by a connecting portion.

In some configurations, the connecting portion is offset from the longitudinal axis.

In some configurations, the medical pole handle comprises an elastomeric sleeve on the grip end portion.

In some configurations, the mount comprises a bracket that comprises a first bracket part and a second bracket part.

In some configurations, the first bracket part comprises the fastening mechanism to engage the mounting end portion.

In some configurations, the fastening mechanism comprises an aperture through which a fastener extends or is capable of extending to engage with the mounting end portion.

In some configurations, the aperture and the mounting end portion comprise complementary surface features to fix the handle body in the two or more orientations.

In some configurations, the fastener is concealed when the bracket is coupled to a pole member of a medical pole.

In some configurations, the second bracket part is hingedly coupled to the first bracket part.

In some configurations, the first bracket part and second bracket part extend around a substantial portion of a perimeter of the pole member when the handle is mounted to the pole member.

In some configurations, the fastening mechanism comprises a spring clip arrangement wherein a spring clip is configured to couple an extension of the first bracket part with the mounting end portion.

In some configurations, the spring clip is configured to lock the orientation of the grip end portion.

In accordance with certain features, aspects and advantages of at least one of the embodiments disclosed herein, a medical pole handle is disclosed, the medical pole handle comprising: a grip end portion; and a pole connection end for releasable connection with a pole member of a medical pole; wherein the handle is orientable about an axis that extends along a length of the medical pole handle.

In some configurations, the medical pole handle is orientable in at least two planes.

In some configurations, the medical pole handle comprises a handle intermediate portion, wherein the longitudinal axis extends through a portion of the handle intermediate portion.

In some configurations, the grip end portion is transverse to the handle intermediate portion.

In some configurations, an end of the handle intermediate portion is connected to an end of the grip end portion by a connecting portion.

In some configurations, the connecting portion is offset from the longitudinal axis.

In some configurations, the medical pole handle comprises an elastomeric sleeve on the grip end portion.

In some configurations, the pole connection end comprises a mount and a fastening mechanism.

In some configurations, the mount comprises a bracket that comprises a first bracket part and a second bracket part.

In some configurations, the first bracket part comprises the fastening mechanism to engage an end of the handle intermediate portion.

In some configurations, the fastening mechanism comprises an aperture through which a fastener extends or is capable of extending to engage with the end of the handle intermediate portion.

In some configurations, the aperture and the mounting end portion comprise complementary surface features to fix the handle body in the two or more orientations.

In some configurations, the fastener is concealed when the bracket is coupled to a pole member of a medical pole.

In some configurations, the second bracket part is hingedly coupled to the first bracket part.

In some configurations, the first bracket part and second bracket part extend around a substantial portion of a perimeter of the pole member when the handle is mounted to the pole member.

In some configurations, the fastening mechanism comprises a spring clip arrangement wherein a spring clip is configured to couple an extension of the first bracket part with the mounting end portion.

In some configurations, the spring clip is configured to lock the orientation of the grip end portion.

In accordance with certain features, aspects and advantages of at least one of the embodiments disclosed herein, a medical pole is disclosed, the medical pole comprising: a pole member; a base with wheels, the base supporting the pole member; and the medical pole handle as outlined above extending outwardly from the pole member.

In some configurations, the base comprises extension legs for locating the wheels.

In some configurations, the medical pole handle extends over one of the extension legs.

In some configurations, the medical pole handle extends in an intended movement direction of the medical pole.

In some configurations, the medical pole handle extends opposite an intended movement direction of the medical pole.

In some configurations, the medical pole is the medical pole according to the first aspect above.

In accordance with certain features, aspects and advantages of at least one of the embodiments disclosed herein, a handle body for use with a medical pole is disclosed, the handle body comprising: a grip end portion and a mounting end portion; the mounting end portion configured to cooperate with a mount to couple the handle to a pole member of a medical pole that has a longitudinal axis such that the handle body can be oriented in two or more orientations relative to the pole member, wherein in one of the orientations the grip end portion extends in a direction that is transverse to the pole member and generally horizontal relative to a ground surface, and is non-intersecting with the longitudinal axis of the pole member.

In some configurations, the orientations are about a longitudinal axis of handle body.

In some configurations, the handle body comprises an intermediate portion between the grip end portion and the mounting end portion, wherein the longitudinal axis extends through a portion of the intermediate portion.

In some configurations, the grip end portion is transverse to the intermediate portion.

In some configurations, an end of the intermediate portion is connected to an end of the grip end portion by a connecting portion.

In some configurations, the connecting portion is offset from the longitudinal axis.

In some configurations, an opposite end of the grip end portion is a free end portion.

In some configurations, the handle body generally has a question mark (‘?’) shape, wherein the mounting end portion corresponds to a base of the question mark shape and the grip end portion corresponds to a top of the question mark shape.

In some configurations, the handle body comprising an elastomeric sleeve on the grip end portion.

It would be desirable to provide an identification system that provides ease of identification of a medical apparatus.

In accordance with certain features, aspects and advantages of at least one of the embodiments disclosed herein, a medical apparatus identification system is disclosed, the medical apparatus identification system comprising: a medical apparatus comprising a pole member; and two or more medical apparatus identification tags, each comprising a clip portion arranged to engage with the pole member, wherein at least a portion of each identification tag comprises an identification feature.

In some configurations, the identification tags are configured such that when they are engaged with the pole member with adjacent edges of the identification tags abutting each other, there is substantially no visible gap between the adjacent edges.

In some configurations, the identification tags are configured such that when they are engaged with the pole member, the identification tags cooperate to extend around substantially an entirety of the periphery of the pole member.

In some configurations, the identification tags are configured such that when they are engaged with the pole member, they cooperate to enable a user to view a portion of each identification tag from any angle around the pole member, when the user views the identification tags at eye level.

In some configurations, the identification feature comprises at least one identification colour.

In some configurations, the identification tags comprise different identification colours.

In some configurations, each identification tag has a single identification colour.

In some configurations, each identification tag comprises more than one identification colour.

In some configurations, at least one of the identification tags comprises a protrusion.

In some configurations, the protrusion is substantially planar.

In some configurations, the protrusion comprises a writing surface.

In some configurations, the protrusion comprises a gripping portion.

In some configurations, the clip portion comprises a body with opposing arms.

In some configurations, the clip portion is configured to extend around substantially an entirety of the periphery of the pole member.

In some configurations, the clip portion has a substantially arcuate inner surface.

In some configurations, the clip portion has a substantially C-shaped cross-section.

In some configurations, the clip portion is configured to snap fit onto the pole member.

In some configurations, the clip portion is configured to engage the pole member in a direction that is substantially transverse to a longitudinal axis of the pole member.

In some configurations, the clip portion is configured to provide audible and/or haptic feedback to a user when the clip portion engages with the pole member.

In some configurations, each arm extends from the body at one end to another end that is proximal to a corresponding end of the other arm.

In some configurations, an edge of each arm that is configured to be positioned adjacent an edge of an arm of an adjacent identification tag, comprises at least one step.

In some configurations, the arms taper from one end to the other end.

In some configurations, opposing edges of each arm taper towards one another towards a center of each arm from one end to the other end.

In some configurations, ends of the arms that are distal the body comprise a straight edge that is substantially parallel to a longitudinal axis of the identification tag.

In some configurations, the arms comprise curved edges.

In some configurations, the identification tags are formed from: rigid material, resilient material, plastic(s), non-plastic rigid material(s), non-plastic resilient material(s), or combinations thereof.

In some configurations, the medical apparatus is a medical pole, and the medical apparatus identification system is a medical pole identification system.

In some configurations, the medical pole comprises one or more retaining features for retaining a medical device and/or a medical device component.

In some configurations, the medical pole comprises one or more retaining features for retaining one or more of a blower, a humidifier, a water bag, a breathing tube, a patent interface, a power supply.

In some configurations, the medical pole comprises a base with wheels.

In some configurations, the pole member is an upright pole member.

In accordance with certain features, aspects and advantages of at least one of the embodiments disclosed herein, a medical apparatus identification tag is disclosed, the medical apparatus identification tag comprising: a clip portion arranged to engage with a pole member of a medical apparatus; and an identification colour for identifying the medical apparatus when in use.

In some configurations, the identification tag has a single identification colour.

In some configurations, the identification tag comprises more than one identification colour.

In some configurations, the identification tag comprises a protrusion.

In some configurations, the protrusion is substantially planar.

In some configurations, the protrusion comprises a writing surface.

In some configurations, the protrusion comprises a gripping portion.

In some configurations, the clip portion comprises a body with opposing arms.

In some configurations, the clip portion is configured to extend around substantially an entirety of the periphery of the pole member.

In some configurations, the clip portion has a substantially arcuate inner surface.

In some configurations, the clip portion has a substantially C-shaped cross-section.

In some configurations, the clip portion is configured to snap fit onto the pole member.

In some configurations, the clip portion is configured to engage the pole member in a direction that is substantially transverse to a longitudinal axis of the pole member.

In some configurations, the clip portion is configured to provide audible and/or haptic feedback to a user when the clip portion engages with the pole member.

In some configurations, each arm extends from the body at one end to another end that is proximal to a corresponding end of the other arm.

In some configurations, an edge of each arm that is configured to be positioned adjacent an edge of an arm of an adjacent identification tag, comprises at least one step.

In some configurations, the arms taper from one end to the other end.

In some configurations, opposing edges of each arm taper towards one another towards a center of each arm from one end to the other end.

In some configurations, ends of the arms that are distal the body comprise a straight edge that is substantially parallel to a longitudinal axis of the identification tag.

In some configurations, the arms comprise curved edges.

In some configurations, the identification tag is formed from: rigid material, resilient material, plastic(s), non-plastic rigid material(s), non-plastic resilient material(s), or combinations thereof.

In some configurations, the medical apparatus is a medical pole, and the medical apparatus identification system is a medical pole identification system.

In some configurations, the pole member is an upright pole member.

In accordance with certain features, aspects and advantages of at least one of the embodiments disclosed herein, a medical apparatus identification tag is disclosed, the medical apparatus identification tag comprising: a clip portion arranged to engage with a pole member of a medical apparatus, the clip portion comprising a body with opposing arms, wherein each arm comprises an edge that is complementary with an edge on the other arm, such that when two of the identification tags are engaged with the pole member with the complementary edges of one of the identification tags abutting with the complementary edges of the other one of the identification tags, there is substantially no visible gap between the abutting complementary edges; wherein at least a portion of the identification tag comprises an identification feature.

In some configurations, the clip portion is configured to extend around substantially an entirety of the periphery of the pole member.

In some configurations, the clip portion has a substantially arcuate inner surface.

In some configurations, the clip portion has a substantially C-shaped cross-section.

In some configurations, the clip portion is configured to snap fit onto the pole member.

In some configurations, the clip portion is configured to engage the pole member in a direction that is substantially transverse to a longitudinal axis of the pole member.

In some configurations, the clip portion is configured to provide audible and/or haptic feedback to a user when the clip portion engages with the pole member.

In some configurations, each arm extends from the body at one end to another end that is proximal to a corresponding end of the other arm.

In some configurations, the edge of each arm that is configured to be positioned adjacent the edge of an arm of an adjacent identification tag, comprises at least one step.

In some configurations, the arms taper from one end to the other end.

In some configurations, opposing edges of each arm taper towards one another towards a center of each arm from one end to the other end.

In some configurations, ends of the arms that are distal the body comprise a straight edge that is substantially parallel to a longitudinal axis of the identification tag.

In some configurations, the arms comprise curved edges.

In some configurations, the identification tag is formed from: rigid material, resilient material, plastic(s), non-plastic rigid material(s), non-plastic resilient material(s), or combinations thereof.

In some configurations, the medical apparatus is a medical pole, and the medical apparatus identification system is a medical pole identification system.

In some configurations, the pole member is an upright pole member.

It would be desirable to provide a medical pole with retaining arrangements for supporting one or more accessories from the medical pole.

In accordance with certain features, aspects and advantages of at least one of the embodiments disclosed herein, a medical pole is disclosed, the medical pole comprising: an upright pole member; a base with wheels, the base supporting the upright pole member; and a first retaining arrangement comprising an upwardly extending hook and a second retaining arrangement comprising a downwardly extending hook, wherein the hooks are spaced apart along the upright pole member.

In some configurations, the retaining arrangements comprise a channel or recess to receive the upright pole member.

In some configurations, the first retaining arrangement comprises an end cap.

In some configurations, the first retaining arrangement comprises a securing mechanism.

In some configurations, the first retaining arrangement is coupled to an end of the upright pole member.

In some configurations, the second retaining arrangement is coupled to an intermediate portion of the upright pole member.

In some configurations, the upwardly extending hook comprises a base portion and an extension portion, and wherein the extension portion of the upwardly extending hook is angled inwardly toward a centre of the first retaining arrangement to provide a relatively narrow opening adjacent a free end of the extension portion of the upwardly extending hook.

In some configurations, the extension portion of the upwardly extending hook comprises the free end and a fixed portion, and wherein the free end of the extension portion of the upwardly extending hook is positioned closer to the centre of the first retaining arrangement than the fixed portion of the extension portion of the upwardly extending hook.

In some configurations, the extension portion of the upwardly extending hook is angled toward the centre of the first retaining arrangement at an angle of more than 0 degrees and up to about 5 degrees.

In some configurations, the downwardly extending hook comprises a base portion and an extension portion, and wherein the extension portion of the downwardly extending hook is angled inwardly toward a centre of the second retaining arrangement to provide a relatively narrow opening adjacent a free end of the extension portion of the downwardly extending hook.

In some configurations, the extension portion of the downwardly extending hook comprises the free end and a fixed portion, and wherein the free end of the extension portion of the downwardly extending hook is positioned closer to the centre of the second retaining arrangement than the fixed portion of the extension portion of the downwardly extending hook.

In some configurations, the extension portion of the downwardly extending hook is angled toward the centre of the second retaining arrangement at an angle of more than 0 degrees and up to about 5 degrees.

In some configurations, the hooks are arranged substantially along the same plane.

In some configurations, the hooks are arranged about the channel.

In some configurations, the medical pole comprises at least two hooks arranged on opposite sides of each retaining arrangement.

In some configurations, the at least two hooks are symmetrical.

In some configurations, each retaining arrangement comprises a primary hook.

In some configurations, the primary hook is positioned a first distance from a center of the respective arrangement.

In some configurations, each retaining arrangement comprises a secondary hook.

In some configurations, the secondary hook is positioned a second distance from the respective primary hook.

In some configurations, the second distance is less than the first distance.

In some configurations, the end of each hook is rounded and/or tapered.

In some configurations, the first retaining arrangement comprises a conduit clip element.

In some configurations, the first retaining arrangement comprises a loop arranged below the upwardly extending hook.

In some configurations, the second retaining arrangement comprises a securing mechanism.

In some configurations, the first retaining arrangement and the second retaining arrangement form a retaining assembly.

Features from one or more embodiments or configurations may be combined with features of one or more other embodiments or configurations. Additionally, more than one embodiment may be used together during a process of respiratory support of a patient.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It should be understood that alternative embodiments or configurations may comprise any or all combinations of two or more of the parts, elements or features illustrated, described or referred to in this specification.

This invention may also be said broadly to consist in the parts, elements and features referred to or indicated in the specification of the application, individually or collectively, and any or all combinations of any two or more said parts, elements or features.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth.

The term ‘comprising’ as used in this specification means ‘consisting at least in part of’. When interpreting each statement in this specification that includes the term ‘comprising’, features other than that or those prefaced by the term may also be present. Related terms such as ‘comprise’ and ‘comprises’ are to be interpreted in the same manner.

As used herein the term ‘(s)’ following a noun means the plural and/or singular form of that noun.

As used herein the term ‘and/or’ means ‘and’ or ‘or’, or where the context allows both.

The invention consists in the foregoing and also envisages constructions of which the following gives examples only.

### BRIEF DESCRIPTION OF THE DRAWINGS

Specific embodiments and modifications thereof will become apparent to those skilled in the art from the detailed description herein having reference to the figures that follow, of which:

FIG. 1 is a rear overhead perspective view of a medical pole.

FIG. 2 is a front overhead perspective view of the medical pole, showing the medical pole supporting exemplary accessories.

FIG. 3 is a plan view of the medical pole.

FIG. 4 is rear overhead perspective view of the movable base of the medical pole.

FIG. 5A is a front overhead exploded perspective view of a first configuration wheel of the movable base.

FIG. 5B is a rear overhead exploded perspective view of the first configuration wheel of the movable base.

FIG. 6 is a sectional perspective view of the first configuration wheel.

FIG. 7A is a side view of components of the first configuration wheel in a first mode.

FIG. 7B is a side view of components of the first configuration wheel in a second mode.

FIG. 7C is a schematic side view of components of the first configuration wheel in an optional third mode.

FIG. 8 is a front overhead perspective view of a second configuration wheel of the movable base.

FIG. 9 is a front overhead exploded perspective view of the second configuration wheel.

FIG. 10 is a rear overhead exploded perspective view of the second configuration wheel.

FIG. 11 is a front overhead sectioned perspective view of the second configuration wheel in a first mode.

FIG. 12A is a side view of components of the second configuration wheel in the first mode.

FIG. 12B is a side view of components of the second configuration wheel in a second mode.

FIG. 13A is a front overhead perspective view showing a medical pole handle mounted in a first downwardly-directed orientation relative to the medical pole.

FIG. 13B is a front overhead perspective view showing the medical pole handle mounted in a second transversely-directed orientation relative to the medical pole.

FIG. 13C is a front overhead perspective view showing the medical pole handle mounted in a third upwardly-directed orientation relative to the medical pole.

FIG. 14A is a plan view of a first configuration medical pole handle.

FIG. 14B is a rear overhead perspective view of the first configuration medical pole handle.

FIG. 15A is a rear overhead exploded perspective view of the first configuration medical pole handle.

FIG. 15B is a front overhead perspective view of part of the fastening mechanism of the medical pole handle.

FIG. 16 is a plan view of a second configuration medical pole handle.

FIG. 17 is a front overhead exploded perspective view of the second configuration medical pole handle.

FIG. 18A is a front overhead perspective view of a third configuration medical pole handle, with the handle in a first transversely-directed orientation relative to the medical pole.

FIG. 18B is a front overhead perspective view of the third configuration medical pole handle, with the handle in a second downwardly-directed orientation relative to the medical pole.

FIG. 19 is a front overhead perspective view showing coupling components of the third configuration medical pole handle.

FIG. 20 is a rear overhead perspective view of a medical apparatus identification system.

FIG. 21A is a front overhead perspective view showing a plurality of first configuration identification tags engaged with a pole member of a medical pole.

FIG. 21B is a rear overhead perspective view showing the plurality of first configuration identification tags engaged with the pole member of a medical pole.

FIG. 22A is a plan view of a first configuration identification tag.

FIG. 22B is a front overhead perspective view of the first configuration identification tag.

FIG. 22C is a rear overhead perspective view of the first configuration identification tag.

FIG. 23A is a rear overhead perspective view showing a plurality of second configuration identification tags engaged with a pole member of a medical pole.

FIG. 23B is a front overhead perspective view showing the plurality of second configuration identification tags engaged with a pole member of a medical pole.

FIG. 24A is a plan view of a second configuration identification tag.

FIG. 24B is a front overhead perspective view of the second configuration identification tag.

FIG. 24C is a rear overhead perspective view of the second configuration identification tag.

FIG. 25A is a rear overhead perspective view showing a plurality of third configuration identification tags engaged with a pole member of a medical pole.

FIG. 25B is a front overhead perspective view showing the plurality of third configuration identification tags engaged with a pole member of a medical pole.

FIG. 26A is a plan view of a third configuration identification tag.

FIG. 26B is a front overhead perspective view of the third configuration identification tag.

FIG. 26C is a rear overhead perspective view of the third configuration identification tag.

FIG. 27 is a rear overhead perspective view of retaining arrangements of the medical pole.

FIG. 28 is a front overhead perspective view of the retaining arrangements of the medical pole.

FIG. 29 is a side view of the retaining arrangements of the medical pole.

FIG. 30 is an opposite side view of the retaining arrangements of the medical pole.

FIG. 31 is a rear view of the retaining arrangements of the medical pole.

FIG. 32 is an overhead perspective view of an upper retaining arrangement.

FIG. 33 is a side view of the upper retaining arrangement.

FIG. 34 is a plan view of the upper retaining arrangement.

FIG. 35 is a front view of the upper retaining arrangement.

FIG. 36 is an overhead perspective view of a lower retaining arrangement.

FIG. 37 is a plan view of the lower retaining arrangement.

FIG. 38 is a side view of the lower retaining arrangement.

FIG. 39 is an opposite side view of the lower retaining arrangement.

FIG. 40 is a front or rear view of the lower retaining arrangement.

FIG. 41 is a side view of an alternative configuration upper retaining arrangement.

FIG. 42 is a side view of an alternative configuration lower retaining arrangement.

### DETAILED DESCRIPTION OF SPECIFIC EMBODIMENTS

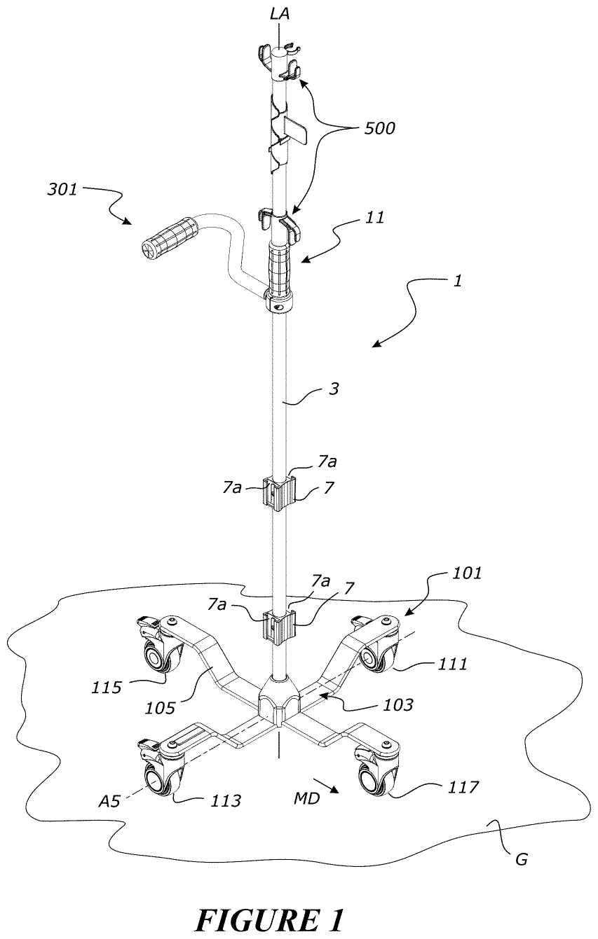

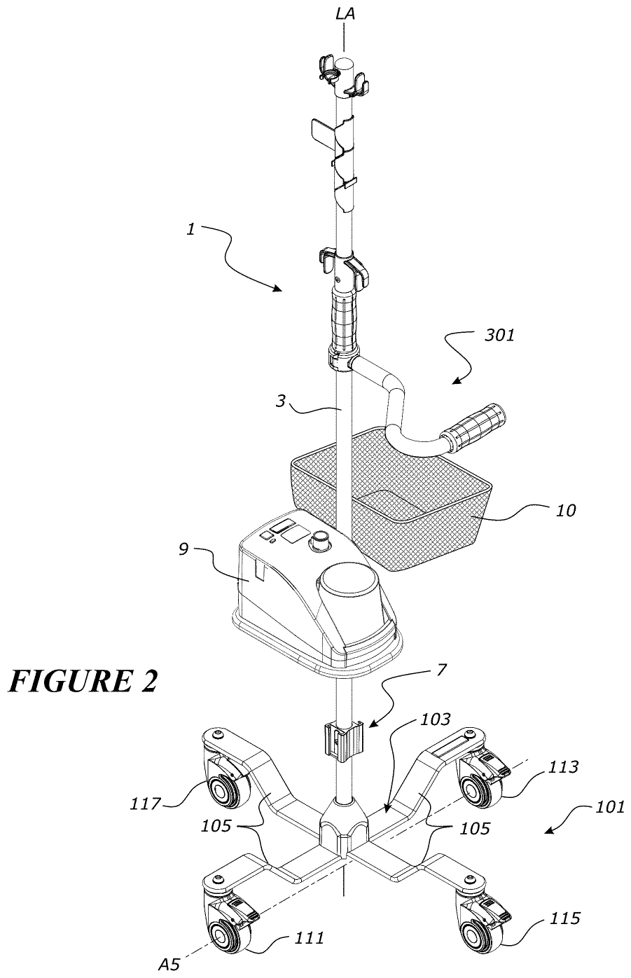

A medical pole 1 for supporting one or more medical accessories is shown in FIGS. 1 and 2.

The medical pole 1 comprises an upright pole member 3 that is supported on a ground surface G by a mobile or movable wheeled base 101.

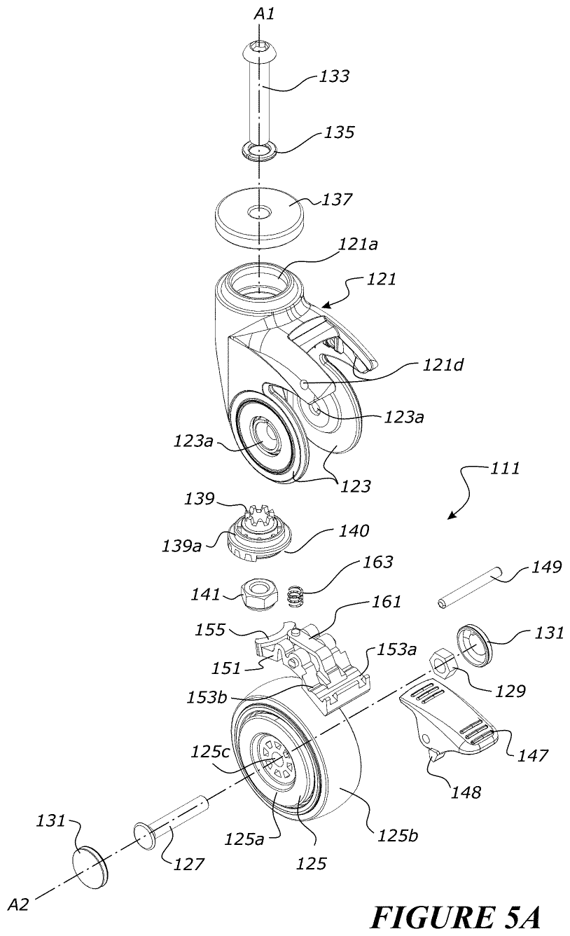

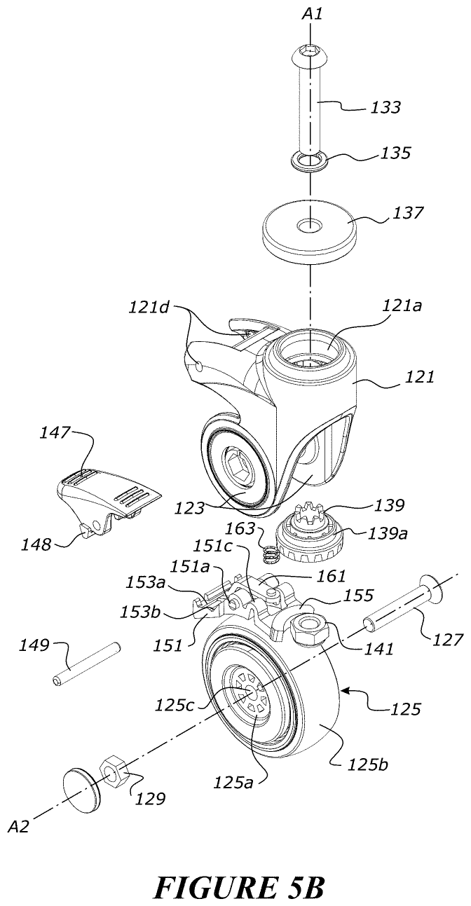

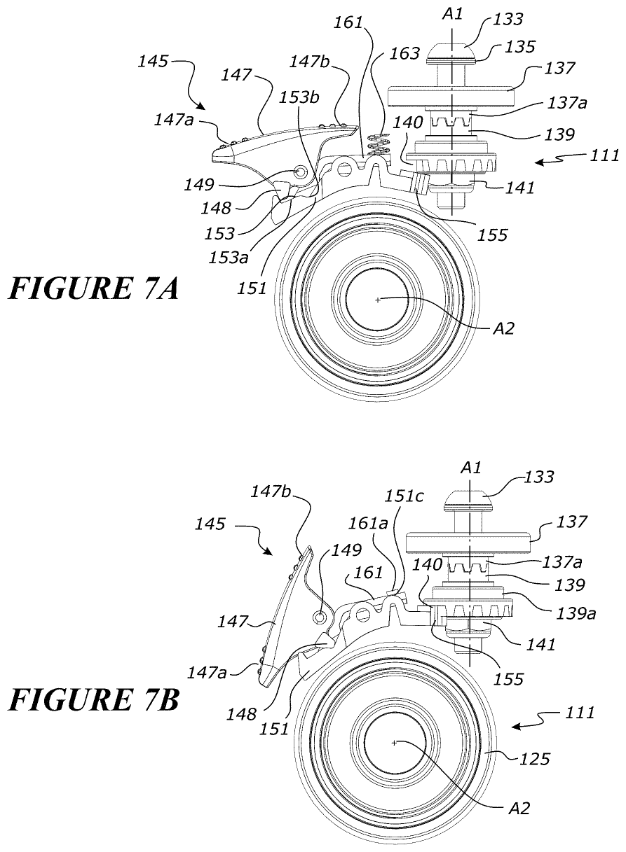

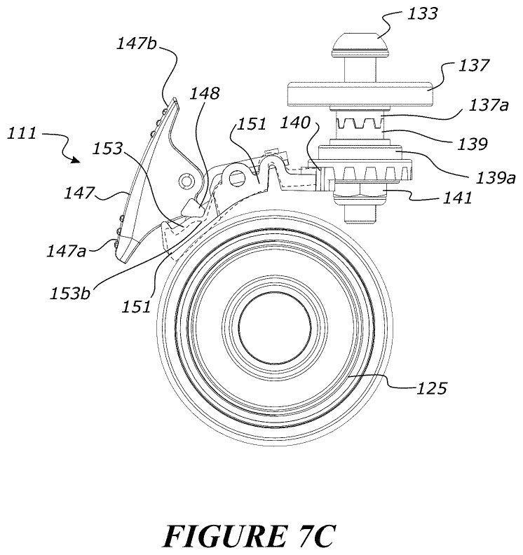

The movable wheeled base 101 comprises a frame 103 and two or more castors or wheels 111, 113, 115, 117. The two or more wheels comprise a first wheel 111 (FIGS. 5A-7C) having a first operational mode (FIG. 7A) in which the first wheel 111 is swivelable about a respective upright axis A1 relative to the frame and is rollable about a respective transverse axis A2, and a second operational mode (FIG. 7B) in which the first wheel 111 is rollable about the respective transverse axis A2 but is fixed or non-swivelable about the respective upright axis A1.

The first wheel 111 can be considered a ‘steering’ wheel, as it locks the swivel orientation of the wheel while enabling rotation of the wheel in the second mode. The first wheel 111 therefore enables but controls movement of the medical pole 1.

The first wheel 111 is configurable or user-selectable between the first mode and the second mode.

When the first wheel 111 is in the second mode, the first wheel 111 maintains a fixed orientation/position about the upright axis A relative to the frame 103 so it cannot swivel.

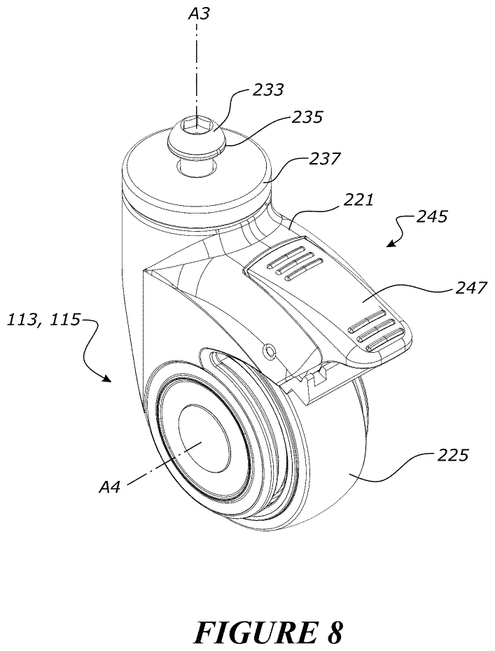

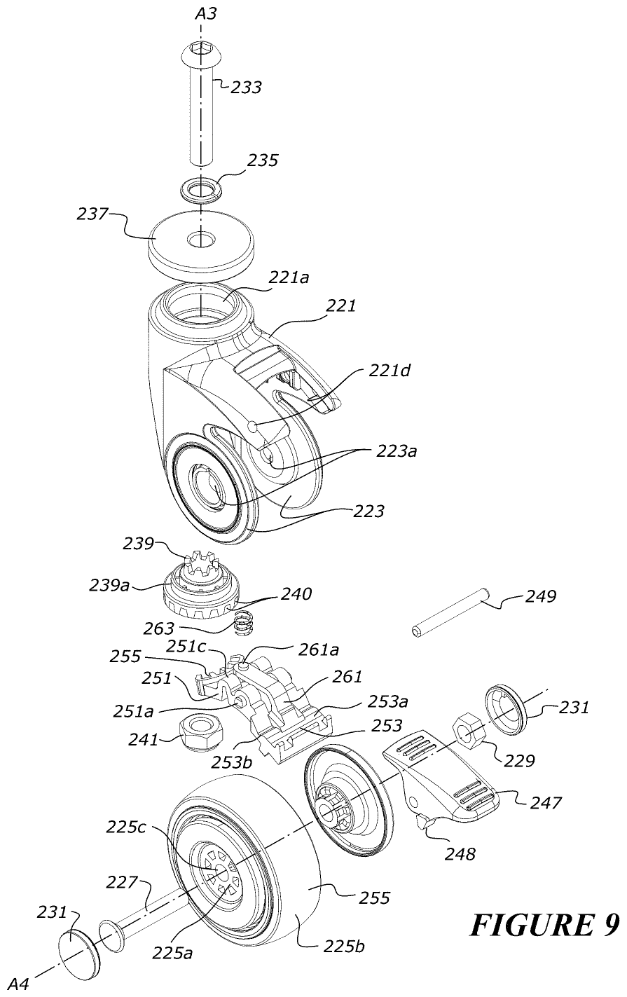

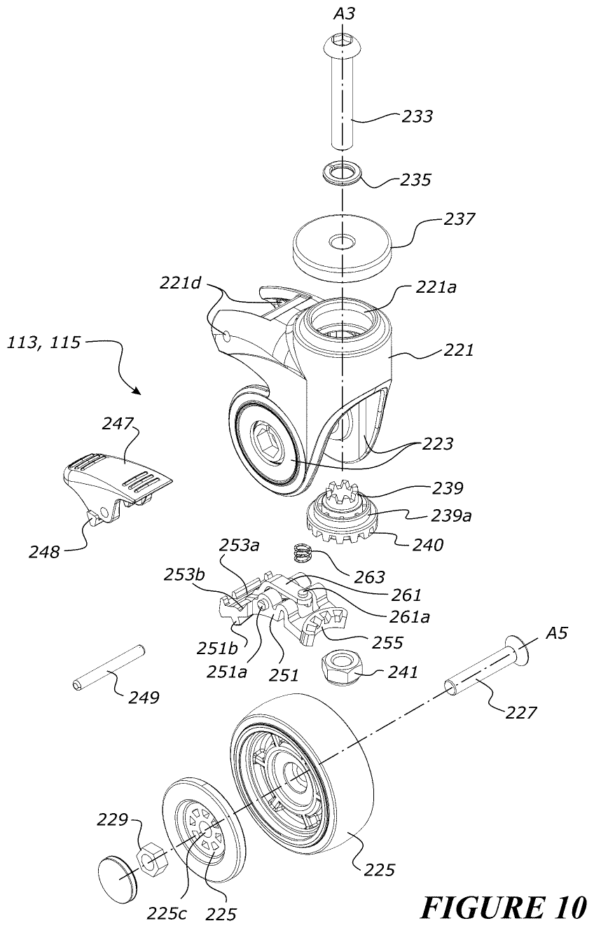

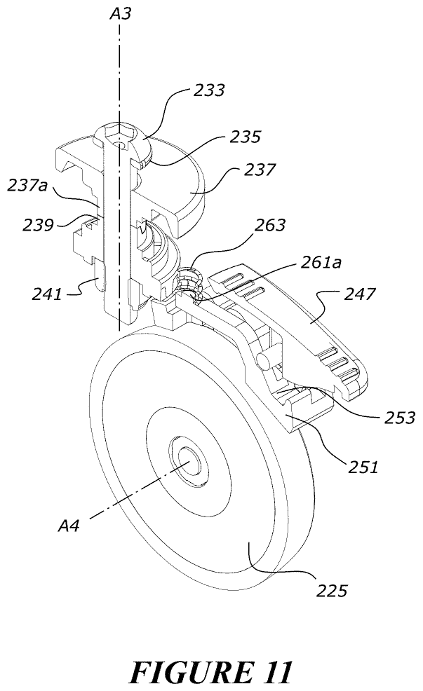

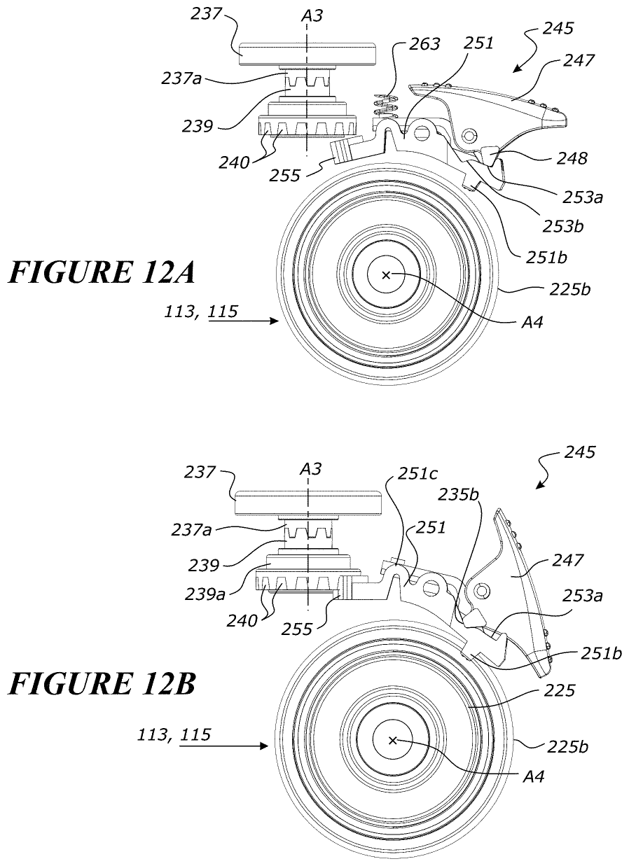

The base 101 comprises a second wheel 113 (FIGS. 8-12B) having a first operational mode (FIG. 12A) in which the second wheel 113 is swivelable about a respective upright axis A3 relative to the frame 103 and is rollable about a respective transverse axis A4, and a second operational mode (FIG. 12B) in which the second wheel is fixed or non-rollable about the respective transverse axis A4 and is fixed or non-swivelable about the respective upright axis A3. The second wheel is configurable between the first mode and the second mode.

The second wheel 113 can be considered a ‘locking’ wheel, as it locks the swivel orientation of the wheel and the rotation of the wheel in the second mode. The second wheel 113 substantially inhibits movement of the medical pole 1.

The second wheel 113 is configurable or user-selectable between the first mode and the second mode.

When the second wheel 113 is in the second mode, the second wheel 113 maintains a fixed orientation/position about the upright axis A3 relative to the frame 103 so it cannot swivel. Additionally, when the second wheel 113 is in the second mode, the wheel is in a braked position, so that the second wheel 113 maintains a fixed position about the transverse axis A4.

If a single wheel of the medical pole is locked so it cannot roll, limited movement of the medical pole on the ground surface G might be possible. In a configuration in which two or more wheels of the medical pole can be locked so they cannot roll, movement of the medical pole on the ground surface G will be prevented.

The first wheel 111 and the second wheel 113 are arranged on the wheeled base substantially opposite each other, with the upright pole member 3 located substantially between the first wheel and the second wheel.

The frame 103 comprises at least one extension leg 105 with a respective one of the wheels 111, 113, 115, 117 arranged along the extension leg 105. In the configuration shown, the frame 103 comprises a plurality of the extension legs 105, each having a respective one of the wheels 111, 113, 115, 117 arranged along the extension leg. The frame 113 may comprise two, three, four, or more extension legs, with a respective wheel positioned along the extension leg.

The wheels 111, 113, 115, 117 may be located at any suitable position along the extension legs 105, such as at an intermediate position or at or adjacent an outer end of the extension legs.

One or more wheels may be provided at a position other than along an extension leg; for example, under a centre of the frame 103.

With reference to FIG. 4, the frame 103 comprises a central hub 107 for connecting to the upright pole member 3. The hub comprises a recess 108 for receipt of a bottom end of the upright pole member 3, to support the upright pole member 3 from the frame 103 of the base 101.

The recess 108 comprises a plurality of engagement features 108a to engage with complementary engagement features on the bottom end of the pole member 3, to inhibit rotation of the pole member 3 relative to the base 101 when the pole member 3 is engaged with the recess 108 of the frame 103. In the form shown, the engagement features 108a comprise a plurality of protrusions that engage with complementary protrusions (not shown) on the bottom end of the pole member 3. Alternatively, the recess 108 and the bottom end of the pole member 3 could have a non-circular cross-section to inhibit rotation of those components relative to each other.

The bottom end of the pole member 3 may engage with the hub 107 of the frame 103 in an interference fit, press fit, or taper lock fit. Alternatively, one or more fasteners could be used to maintain the upright pole member 3 in engagement with the base 101. In that configuration, the frame 103 may not have a central hub 107. The upright pole member 3 could be integrally formed with the base 101.

The extension legs 105 extends outwardly from the centre of the frame 103 perpendicularly relative to the upright pole member 3. In the configuration shown, the extension legs 105 extend radially outwardly from the centre of the frame 103.

In the configuration shown, inner proximal portions 105a of the extension legs 105 extend horizontally outwardly from the centre of the frame 103. Outer distal portions 105c extend outwardly and parallel relative to the inner proximal portions 105a, but are positioned higher than the inner proximal portions 105a to provide clearance for the wheels 111, 113, 115, 117. This enables the bottom end of the upright pole member 3 to be positioned closer to the ground surface G, lowering the centre of gravity and enhancing the stability of the upright pole member 3 and supported accessories.

An angled intermediate portion 105b extends outwardly and upwardly at a non-horizontal and non-vertical angle, between the inner proximal portions 105a and the outer distal portions 105c. Alternatively, the intermediate portion 105b could extend vertically to provide a more stepped configuration of the extension legs 105.

In an alternative configuration, the extension legs 105 may be angled to extend outwardly and upwardly from the centre of the frame 103 to the connection to the wheels 111, 113, 115, 117, and may be substantially straight along their lengths other than adjacent the outer distal ends that connect to the wheels.

FIGS. 5A to 7C show details of the first wheel 111.

The first wheel 111 comprises a body member 121.

The body member 121 defines an upwardly open aperture 121a for receiving wheel mounting components for mounting the wheel 111 to the frame 103.

A lower portion of the body member 121 comprises a pair of spaced apart vertically oriented flanges 123. The spacing between the flanges 123 is configured to enable a wheel member 125 to be rotatably received between the flanges 123.

The wheel member 125 comprises a hub 125a and a peripheral rolling surface 125b. The rolling surface 125b comprises a suitable material to provide a smooth rolling action on the ground surface G, such as natural or synthetic rubber or an elastomer for example.

A pin 127 extends through apertures 123a in the flanges 123 and aperture 125c in the hub 125a to rotatably mount the wheel between the flanges 123. A nut 129 holds the pin 127 in place.

Aesthetic caps 131 cover the pin 127, nut 129, and centre of the hub 125a.

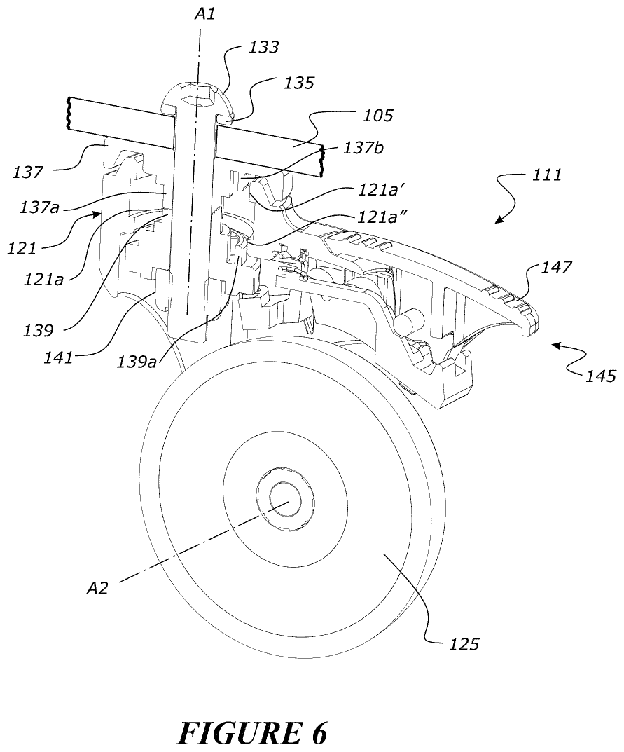

With reference to FIGS. 5A, 5B, and 6, the wheel mounting components for mounting the wheel 111 to the frame 103 comprises a fastener 133, a washer 135, a coupler member or cap 137 to cover the upwardly open aperture 121a, a positive engagement coupler 139, and nut 141. The positive engagement coupler 139 engages with a complementary positive engagement member 137a that extends downwardly from an underside of the cap 137. When the positive engagement coupler 139 is engaged with the positive engagement member 137a, relative rotation between those two components is prevented.

In the configuration shown, the positive engagement member 137a and positive engagement coupler 139 each comprise a plurality of complementary teeth and recesses, but could alternatively have a different configuration such as one tooth and one recess for example.

To mount the wheel 111 to the frame 103, the wheel is positioned beneath the corresponding extension leg 105 of the frame, and fastener 133 is inserted through an aperture in the frame and through the cap 137, body 121, positive engagement member 137a, and positive engagement coupler 139, and the nut 141 is tightened on the fastener 133. That maintains the positive engagement coupler 139 in engagement with the positive engagement member 137a, so that relative rotation of those two components is prevented.

When the wheel 111 is in the first mode, the body 121 is swivelable about the upright axis A1 relative to the positive engagement member 137a and positive engagement coupler 139, and thereby relative to the frame 103 of the movable base 101.

It can be seen from FIG. 6 that radial clearance is provided between upper and lower peripheral walls 121a′, 121a″ of the body aperture 121a, and corresponding annular members 137b, 139a on the cap 137 and the positive engagement coupler 139, to enable that swiveling.

The first wheel 111 has a locking mechanism 145 to enable a user to select the two modes of the wheel; i.e. the first mode in which the first wheel 111 is swivelable about the respective upright axis A1 relative to the frame and is rollable about the respective transverse axis A2, and the second operational mode in which the first wheel 111 is rollable about the respective transverse axis A2 but is fixed or non-swivelable about the respective upright axis A1.

The locking mechanism 145 comprises a user actuation member 147.

The user actuation member 147 is in the form of a lever. In the configuration shown, the user actuation member is in the form of a depressible lever.

The user actuation member 147 can be actuated by the user's foot which means the user does not need to bend and can instead simply actuate the lever (pedal) using their right or left foot. The independent user actuation member 147 may be a biased lever and allows the wheel 111 to operate in two, or optionally three, modes. Other forms of actuator may be used, for example a hand actuator which has an arm that a user may manipulate to lock actuate the locking mechanism.

In the first mode (FIG. 7A), the locking mechanism 145 is in its unlocked position (when a biased lever is used this may be the default position) in which both the swivel and rolling movement of the wheel is unlocked and the steering wheel can roll over the ground surface G as well as swivel in any direction freely.

Actuating the locking mechanism 145 to a locked position, places the steering wheel in the second mode (FIG. 7B) in which the swivel movement of that wheel is locked and therefore the wheel can still roll but it cannot swivel. This second mode is particularly desired when the medical pole movement is desired in a particular direction. Whilst the steering wheel is locked it will roll in that direction irrespective of the swivel movement of other wheels and the lateral movement of the medical pole towards the left/right hand side is prevented or inhibited.

Optionally, as discussed below, in a different embodiment, both the rolling and swivelling of the steering wheel may also be locked by moving the locking mechanism through a third displacement so the wheel is in a third mode (FIG. 7C) in which a wheel brake is engaged.

The user actuation member 147 is pivotally mounted to the wheel body 121 via a transverse pin 149 that extends through apertures 121d in the wheel body 121.

An elongate locking member 151 is pivotally mounted to the wheel body 121. The locking member 151 comprises a pair of outwardly extend projections 151a that are received in apertures in the wheel body 121.

The locking member 151 has an underside the generally follows the shape of the wheel member, as shown in side view in FIG. 7A.

An outer end of the locking member 151 has a recess 153 with an outer end 153a having a small vertical dimension and an inner end 153b having a large vertical dimension. The inner end 153b of the recess is provided by a wedge-shaped recess section that corresponds generally to the shape of a wedge-shaped engagement projection 148 that extends downwardly from an underside of the user actuation member 147. The wedge-shaped engagement projection 148 is positioned adjacent the pivot of the user actuation member 147, toward the outer end of the locking member 151.

A biasing lever 161 is coupled to the locking member 151.

An outer end of the biasing lever 161 is connected to the locking member 151, adjacent to the wedge-shaped portion 153b of the recess 153.

An inner end of the biasing lever 161 interacts with a biasing member 163. In the configuration shown, the biasing member 163 is spring. The biasing member could be any suitable type of member, such as compression spring, tension spring, leaf spring, or resilient block for example.

The inner end of the biasing lever 161 is biased downwardly by the biasing member 163, which biases the outer end of the locking lever 151 upwardly and the inner end of the locking lever 151 downwardly.

The locking mechanism 145 comprises a tooth/teeth and slot(s) arrangement. The user actuation member 147 is configured to actuate the tooth/teeth and slot(s) toward one another for engagement to lock swiveling of the first wheel 111.

The inner end of the locking member 151 comprises an engagement feature 155 to engage with a complementary engagement feature 140 that is fixed in position relative to the frame 103 of the movable base 101, when the first wheel 111 is in the second mode. The engagement feature 155 is disengaged from the complementary engagement feature 140 when the first wheel 111 is in the first mode.

The first wheel 111 has a fixed orientation about the respective upright axis A1 relative to the upright pole member 103 in the second configuration mode.

In the configuration shown, the engagement feature 155 comprises a single arcuate member and the complementary engagement feature 140 comprises a complementary arcuate recess. The single arcuate member 155 acts as a tooth and the complementary arcuate recess acts as a receiver slot. The engagement features could have different configurations, such as a differently-shaped projection that is received in a complementary recess for example.

The complementary arcuate recess 140 is provided in a peripheral wall in an underside of the positive engagement coupler 139. Alternatively, the recess 140 could be provided in a different member that is fixed relative to the frame 103, such as the cap 137 for example.

In the described configuration, the locking mechanism 145 comprises an upper portion (e.g. complementary engagement feature 1401 that is selectively engageable with a bottom portion (e.g. engagement feature 155) to lock swivel of the first wheel 111. Alternative configurations or orientations could be used.

The locking mechanism 145 enables a user to select the two modes of the first wheel 111. When the user wants the first wheel to swivel freely about upright axis A1 and rotate or roll freely about transverse axis A2 (i.e. unlock the wheel or put it in the first mode), the locking mechanism 145 is in the position shown in FIG. 7A. The wedge-shaped engagement projection 148 of the user actuation member 147 is at the outer end 153a of the recess 153, and the engagement feature 155 is biased out of engagement from the complementary engagement feature 140 by the biasing member 163. Due to the biasing, this is the default mode.

To lock swivel but enable free rotation or rolling of the first wheel 111 (i.e. put the wheel in the second mode), the user applies downward force to an outer portion 147a of the user actuation member 147. That moves the wedge-shaped engagement projection 148 into the inner end 153b of the recess, which forces the outer end of the locking member 151 down and moves engagement feature 155 into engagement with the complementary engagement feature 140, against the force of the biasing member 163. Swivelling of the first wheel 111 about the upright axis A1 is prevented by that engagement. Rolling of the wheel is unimpeded so that the wheel can roll freely when external force is applied to the medical pole.

When the user wants to reselect the first mode, they apply force against an inner portion 147b of the user actuation member 147. The user could also interact with the user actuation member 147 in a different way, such as by applying upward force to an underside of the outer portion 147a of the user actuation member for example. That tilts the user actuation member 147 upwardly until the wedge-shaped engagement projection 148 clears the inner end 153b of the recess. The wedge-shaped engagement projection 148 can then slide outwardly to the outer end 153a of the recess, while the biasing member 163 causes the outer end of the locking member 151 to lift and the inner end of the locking member to lower, so that the engagement feature 155 moves out of engagement from the complementary engagement feature 140.

In the configuration shown, the first wheel 111 has one user-selectable fixed orientation position about the respective upright axis A1.

Alternatively, the first wheel 111 may have a plurality of user-selectable fixed-orientation positions about the respective upright axis A1. For example, the first wheel 111 may have four or more user-selectable fixed orientation positions about the respective upright axis A1. For example, the first wheel may have six or eight user-selectable fixed-orientation positions about the respective upright axis A1.

The user-selectable fixed orientation positions may be substantially orthogonal to each other.

A plurality of user-selectable fixed orientation positions may be provided by having angularly spaced engagement features that are selectively engageable with a plurality of angularly spaced complementary engagement features. For example, engagement feature 155 may comprise a plurality of angularly spaced teeth and the complementary engagement feature 140 may comprise a plurality of angularly spaced complementary teeth or recesses.

At least one of the user-selectable positions is parallel to an intended movement direction MD of the medical pole 1, as shown in FIG. 1.

In some configurations, the first wheel 111 is able to move into the one or more user-selectable fixed orientation positions if it is not in such position when the locking mechanism of the first wheel is activated.

A resiliently flexible kinked region or bump 151c (FIGS. 5B and 7B) in the elongate locking member 151 will keep the user actuation member 147 actuated and the engagement feature 155 biased upwardly so that it will engage with the complementary engagement feature when the first wheel swivels into the fixed orientation position.

In some configurations, the first wheel 111 may have a user selectable third operational mode in which the first wheel 111 is non-rollable about the respective transverse axis A2 and is non-swivelable about the respective upright axis A1. The first wheel is configurable or user-selectable between the first mode, the second mode, and the third mode.

The third mode is schematically shown in broken lines in FIG. 7C. In the third mode, the outer portion of the locking member 151 has been driven further downwardly by further downward movement of the user actuation member 147, so that a lower surface of the locking member 151 has engaged with the wheel member 125 to prevent the wheel rotating. To enable that to occur, clearance may be provided between the engagement features 140, 155 when the wheel is in the second mode, so that the engagement feature 155 can move further upward relative to the complementary engagement feature 140 when the wheel is configured from the second mode to the first mode. The outer portion of the locking member 151 may have, or be in the form of, a brake pad.

The recess 153 may be provided with a third section further inward than the second section 153b, with the wedge-shaped member 148 received in the third section in the third mode.

FIGS. 8 to 12B show the second wheel 113 of the movable base 101. Unless described below, the features, functioning, and options for the second wheel 113 are the same as for the first wheel 111. Like numbers indicate like parts with the addition of 100.

The second wheel 113 has a first operational mode (FIG. 12A) in which the second wheel 115 is swivelable about a respective upright axis A3 relative to the frame 103 and is rollable about a respective transverse axis A4, and a second operational mode (FIG. 12B) in which the second wheel is fixed or non-rollable about the respective transverse axis A4 and is fixed or non-swivelable about the respective upright axis A3, wherein the second wheel is configurable between the first mode and the second mode. Unlike the first wheel 111, the second wheel 113 does not have a mode in which swivel is disabled but rotation is enabled.

The second wheel 113 is configurable or user-selectable, via the locking mechanism 245, between the first mode and the second mode.

In the first mode, the locking mechanism 245 is in its unlocked position (when a biased lever is used this may be the default position) in which the second wheel 113 is swivelable about the respective upright axis A3 relative to the frame 103 and is rollable about a respective transverse axis A4 and the second wheel 113 can roll over the ground surface G as well as swivel in any direction freely.

Actuating the locking mechanism 245 to a locked position, places the second wheel 113 in the second mode in which both the swivel movement of that wheel is locked and rotation or rolling movement of that wheel is locked. In that mode, the second wheel 113 is fixed or non-rollable about the respective transverse axis A4 and is fixed or non-swivelable about the respective upright axis A3.

When the second wheel 113 is in the second mode, the second wheel 113 maintains a fixed orientation/position about the upright axis A3 relative to the frame 103 so it cannot swivel. Additionally, when the second wheel 113 is in the second mode, the wheel is in a braked position, so that the second wheel 113 maintains a fixed position about the transverse axis A4.

An underside of an outer portion of the locking member 251 has a brake member 251b that engages against the rolling surface 225b of the wheel member 225, to prevent rotation of the second wheel 113 about the transverse axis A4 in the second mode (FIG. 12B). As shown in FIG. 12A, the brake member 251b is clear of the rolling surface 225b of the wheel in the first mode.

The brake member 251b may be in the form of a brake pad.

The brake member 251b is actuatable by the user actuation member 247 to engage the rolling surface 225b of wheel 225 that contacts the ground surface G when in use.

The second wheel 113 has a plurality of user-selectable fixed-orientation positions about the respective upright axis A3. For example, the second wheel 113 may have four or more user-selectable fixed orientation positions about the respective upright axis A3. For example, the second wheel may have six or eight user-selectable fixed-orientation positions about the respective upright axis A3.

The user-selectable fixed orientation positions may be substantially orthogonal to each other.

A plurality of user-selectable fixed orientation positions is provided by having angularly spaced engagement features that are selectively engageable with a plurality of angularly spaced complementary engagement features. The engagement feature 255 comprises a plurality of angularly spaced teeth and the complementary engagement feature 240 comprises a plurality of angularly spaced complementary teeth or recesses.

In the first mode (FIG. 12A), the locking mechanism 245 is in the default position in which the teeth or recesses of the complementary engagement feature 240 do not engage with the complementary teeth of the engagement feature 255 and therefore the wheel may swivel freely. Similarly, the brake member 251b does not contact or engage with the wheel member 225 so that the wheel can also roll freely when external force is applied to the medical pole.

In the second mode (FIG. 12B), the locking mechanism 245 is displaced to a second position so that the teeth or recesses of the complementary engagement feature 240 engage with the teeth of the engagement feature 255 and therefore the locking wheel is prevented from swivelling. Also, when the locking mechanism is displaced to the second position, the brake member 251b at or near one end of the second portion engages with the wheel member 225 so that the rolling or rotating of the wheel member 225 is prevented or inhibited.

In an alternative configuration, the second wheel 113 may have a single user-selectable fixed orientation position.

The movable base 101 may comprise a third wheel 115 located at a position generally between and offset from the first wheel 111 and the second wheel 113. The third wheel 115 may be located at a position generally between and orthogonal to the first wheel 111 and the second wheel 113. The third wheel 115 is adjacent to the second wheel 113.

In the configuration shown, the third wheel 115 is the same type of wheel as the second wheel 113 (i.e. a locking wheel). That is, the third wheel 115 has a first operational mode in which the third wheel 115 is swivelable about a respective upright axis relative to the frame 103 and is rollable about a respective transverse axis, and a second operational mode in which the third wheel is non-rollable about the respective transverse axis and is non-swivelable about the respective upright axis, wherein the third wheel is configurable between the first mode and the second mode. The features, functioning, and operation of the third wheel 115 are the same as for the second wheel 113.

The second wheel 113 and third wheel 115 can be moved between their first and second modes independently of each other.

Actuating the locking mechanisms 245 of the second wheel 113 and third wheel 115 to their locked positions, places each wheel in the second mode in which both the swivel as well as rolling movement of that wheel is locked and therefore the wheel can neither roll nor swivel. This second position is generally desirable when the medical pole 1 is desired to be in stationary position because if two adjacent locking wheels of the four-wheel medical pole cannot swivel or roll then the medical pole cannot be moved easily.

In the described configuration, the first wheel 111 comprises fewer teeth 155 and slots 140 compared to the teeth 255 and slots 240 of the second wheel 113 and/or the third wheel 115. This allows the first wheel 111 to orient to fewer angular positions than the second wheel 113.

The movable base 101 may comprise a fourth wheel 117. The fourth wheel 117 may be provided at any suitable location on the movable base 101. In some configurations, the fourth wheel 117 is located generally between and offset from the first wheel 111 and the second wheel 113, generally between and offset from the second wheel 113 and the third wheel 115, or generally between and offset from the third wheel 115 and the first wheel 111. The fourth wheel may be located substantially on an opposite side of the base to the third wheel 117 as shown. The fourth wheel 117 has a single operational mode in which the fourth wheel is swivelable about a respective upright axis relative to the frame and is rollable about a respective transverse axis.

In the first configurations of the wheels 111, 113, 115, and in the single configuration of the wheel 117, the wheels have the ability to roll and swivel freely. That facilitates movement of the medical pole 1 on the ground surface G. Additionally, the locking wheels 113, 115 can lock both the roll and the swivel of the wheels, and the steering wheel 111 can lock the swivel of the wheel to a particular direction but not the roll.

FIGS. 1 and 2 show an exemplary configuration of the wheels 111, 113, 115, 117 on the movable base 444101. In an alternative exemplary configuration, the position of the third wheel 115 and the fourth wheel 117 could be swapped.

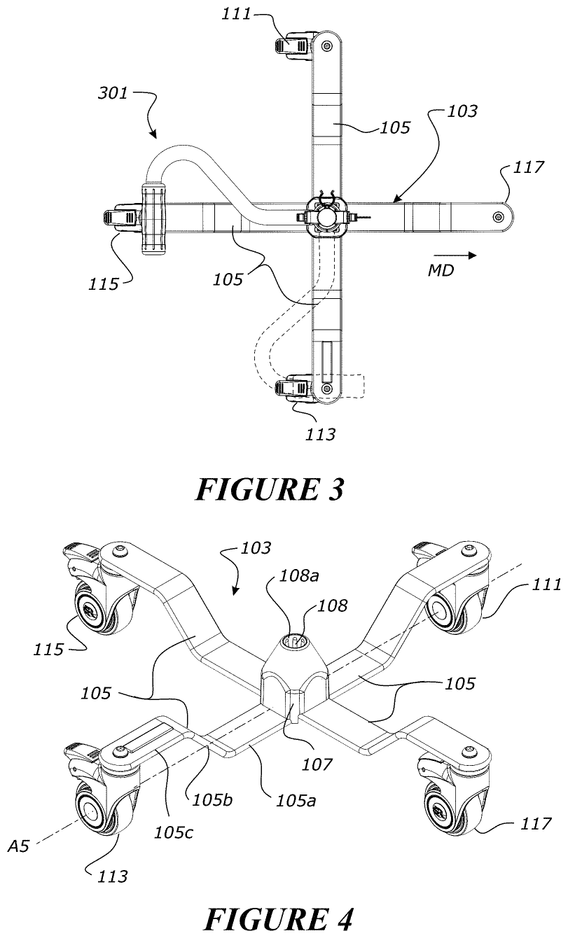

The first wheel 111 is a steering wheel with a lockable swivel which restricts lateral movement (which is perpendicular to the direction of travel) of the medical pole 1 due to the creation of a predominant direction of travel MD. The wheel 111 can still roll in its locked position. The alignment of the first wheel 111 is parallel to the direction of travel MD shown in FIG. 3.

The user will typically push/pull the medical pole 1 by use of a medical pole handle 301. The handle 301 is positioned above the third wheel 115 position in FIG. 2 (if the user is pushing in the direction MD indicated), in such a case the steering wheel may be in the position shown for wheel 111 or wheel 113. Less desirable would be the steering wheel in the position shown for wheel 117 and the least desirable embodiment would be the steering wheel in the position shown for wheel 115.

Two locking wheels 113, 115 which lock both the swivel and the roll allow the medical pole 1 to rest on an inclined plane without pivoting/skidding around a singular point. The positions of the locking wheels 113, 115 within the assembly are of less importance than the position of the steering wheel 111.

The wheels 111, 113, 115, 117 may be evenly distributed radially with respect to the centre of the medical pole.

In an alternative configuration, the movable base 101 comprise only three wheels; two locking wheels and one steering wheel.

In an alternative configuration, the movable base 101 comprises more than four wheels with at least one steering wheel and at least two locking wheels.

In an alternative configuration, the steering wheel 111 includes the third mode that locks the swivel and roll, in which case one fewer locking wheels may be used.

The fourth wheel 117 is a normal wheel that can swivel and roll freely and does not have a user actuator or locking mechanism. That wheel could be replaced with a locking wheel or a steering wheel, however that would add to complexity and cost.

For example, the fourth wheel 117 may have a first mode in which the fourth wheel is swivelable about a respective upright axis relative to the frame 103 and is rollable about a respective transverse axis, and a second mode in which the fourth wheel is non-rollable about the respective transverse axis and is non-swivelable about the respective upright axis, wherein the fourth wheel is configurable between the first mode and the second mode.

With such a configuration, the user can lock the rolling and swiveling of three out of four wheels.

The external appearance of the steering wheel 111 may be the same as that of the locking wheels 113, 115. In some configurations, the actuator 147 of the steering wheel 111 is of different colour than the actuator 247 of each locking wheel so that the steering wheel is visually distinctive from the locking wheels.

In a hospital or homecare environment, the medical pole 1 may need to be portably transported to various locations. A purpose of this exemplary configuration is to provide a medical pole 1 that has a lockable wheel arrangement to prevent movement whenever necessary and to provide easy steering, manoeuvring, and control of the medical pole 1 for effective transportation. The purpose is to also provide a simple, inexpensive and efficient locking mechanism and arrangement for one or more of the wheels.

The upright pole member 3 comprises one or more retaining features 7 for retaining an accessory such as a medical device and/or a medical device component. In the configuration shown in FIGS. 1 and 2, the upright pole member 3 comprises two retaining features 7 that are spaced apart along the upright pole member 3. In another configuration, the upright pole member 3 comprises three or more retaining features 7 that are spaced apart along the upright pole member 3.

In the configurations shown, each retaining feature 7 has two receiver portions 7a that are configured to receive complementary coupling portions on medical devices or medical device components.

The receiver portions 7a are provided on opposite sides of the retaining feature 7. That way, each retaining feature 7 can support a medical device or a medical device component on either side of the upright pole member 3. In alternative configurations, one or more of the retaining features 7 may have a single receiver portion 7a or may have three or more receiver portions 7a.

The receiver portions 7a comprise upwardly open recesses that are configured to receive downwardly projecting tongues on a medical device or a medical device component. The receiver portions 7a could alternatively have a different configuration, such as a projection that is received in a corresponding slot on the medical device, medical device component, or accessory for example.

FIG. 2 shows a medical device in the form of a breathing assistance apparatus 9 and a basket 10 supported from one of the retaining features 7. The retaining features 7 could be used to support any other suitable medical device, medical device component, accessory, or combinations thereof.

The one or more retaining features 7 may for example be for retaining one or more of a blower, a humidifier, a water bag, a breathing tube, a patent interface, a power supply.

The medical pole 1 comprises a medical pole handle 301 extending from the upright pole member 3. The medical pole handle 301 is shown coupled to the upright pole member 3 in FIGS. 1 and 2. Various configurations of the medical pole handle 301 are shown in FIGS. 13A to 18B.

In the configuration shown, the medical pole handle 301 extends from the upright pole member 3 in a direction that is substantially perpendicular to an axis A5 or plane extending through the first wheel 111 and the second wheel 113, when the first and second wheels 111, 113 are oriented in an intended movement direction MD of the medical pole 1.

The medical pole handle 301 is adjustable between multiple angular positions relative to the upright pole member 3.

The medical pole handle 301 may extend from the upright pole member 3 in the intended movement direction MD of the medical pole 1. Alternatively, the medical pole handle 301 may extend opposite to the intended movement direction MD of the medical pole 1.

In the configuration shown, the medical pole handle 301 extends from the upright pole member 3 in a direction away from the first wheel 111.

The medical pole handle 301 could extend from the upright pole member 3 in any suitable direction away from the first wheel 111. The medical pole handle 301 could, for example, extend in a direction over one of the extension legs 105 away from the first wheel 111 (e.g. over the extension leg 105 associated with the second wheel 113, third wheel 115, or fourth wheel 117), or could extend away from the first wheel 111 at an angle that is between two of the extension legs 105.

In the configuration shown, the medical pole handle 301 extends in a direction away from the first wheel that is transverse or orthogonal to the first wheel. Alternatively, the medical pole handle 301 could extend in a direction away from the first wheel 111 that is opposite to the first wheel 111, as shown in broken lines in FIG. 3 for example.

Having the medical pole handle 301 extending in the direction shown in FIGS. 1 and 2 allows all of the user-actuable wheels 111, 113, 115, to be easily accessible by a user while accessing the medical pole handle 301. In an alternative configuration, the positions of the second wheel 113 and the third wheel 117 could be swapped.

The medical pole handle 301 extends from the upright pole member 3 in a direction that is substantially parallel to and over an extension leg 105.

Referring to FIGS. 13A to 15B, the medical pole handle 301 is an assembly comprising a handle body 303 with a grip end portion 305 and a mounting end portion 307.

A mount 309 couples the handle body 303 to the upright pole member 3 of the medical pole 1.

The medical pole handle 301 is configured to be removably mounted to the upright pole member 3 of the medical pole 1 by the mount 309.

A fastening mechanism 313 couples the handle body 303 with the mount 311 in two or more orientations with respect to the mount 311.

The orientations are about a longitudinal axis A6 of handle body 303.

The medical pole handle 301 is orientable about the axis A6 that extends along a length of the medical pole handle 301.

The axis A6 extends along the mounting end portion 307 and across the grip end portion 305.

The medical pole handle 301 is rotatable about the axis A6.

The handle body 303 is orientable in at least two planes.

In some configurations, the two planes are transverse to each other.

In one of the orientations (FIG. 13B) the grip end portion 305 extends in a direction D that is transverse to the longitudinal axis LA of the upright pole member 3 and generally horizontal relative to a ground surface G, and that is non-intersecting with the longitudinal axis LA of the pole member. In this configuration, the direction D and the longitudinal axis LA form skew lines (lines that do not intersect and are not parallel).

In another of the orientations (FIG. 13A) the grip end portion 305 extends substantially parallel to the longitudinal axis LA of the upright pole member 3 and generally perpendicularly relative to the ground surface G. In this configuration, the grip end portion 305 extends downwardly.

In another of the orientations (FIG. 13C) the grip end portion 305 extends substantially parallel to the longitudinal axis LA of the upright pole member 3 and generally perpendicularly relative to the ground surface G. In this configuration, the grip end portion 305 extends upwardly.

In another of the orientations (not shown) the grip end portion 305 extends transversely to the longitudinal axis LA of the upright pole member 3 and generally horizontally relative to a ground surface G, in an opposite direction to FIG. 13B.

The handle body 303 may be orientable in two, three, or four of these orientations.

The handle body 303 comprises an intermediate portion 308 between the grip end portion 305 and the mounting end portion 307. The longitudinal axis A6 extends through a portion of the intermediate portion 308.

The intermediate portion 308 comprises a first inner section 308a adjacent the mounting end portion 307 that is coaxial with the longitudinal axis A6 of the handle body 305.

A second middle section 308b of the intermediate portion extends outwardly from the first inner section 308a at a non-perpendicular and non-parallel angle.

A third outer connecting portion 308c of the intermediate portion 308 is connected to an end of the grip end portion 305. The connecting portion 308c is offset from the longitudinal axis A6.

An opposite end of the grip end portion 305 is a free end portion.

The connecting portion 308c has an arcuate shape.

The grip end portion 305 is transverse to the intermediate portion 308. In the configuration shown, the grip end portion 305 extends substantially perpendicularly relative to the first inner section 308a of the intermediate portion 308.

The handle body 303 generally has a question mark (‘?’) shape, wherein the mounting end portion 307 and the first inner section 308a of the intermediate portion 308 corresponds to a base of the question mark shape and the grip end portion 305 corresponds to a top of the question mark shape. This provides an ergonomic shape for ease of manipulating the direction of travel of the medical pole 1 in use.

The handle body 303 may be a single integrally formed piece or alternatively could comprise a plurality of members that are connected together.

The handle body 303 comprises an inner linear section 307, 308a that provides an offset distance from the upright pole member 3. The third outer connecting portion 308c aligns the midpoint of the grip 305a with the midpoint of the mount 309.

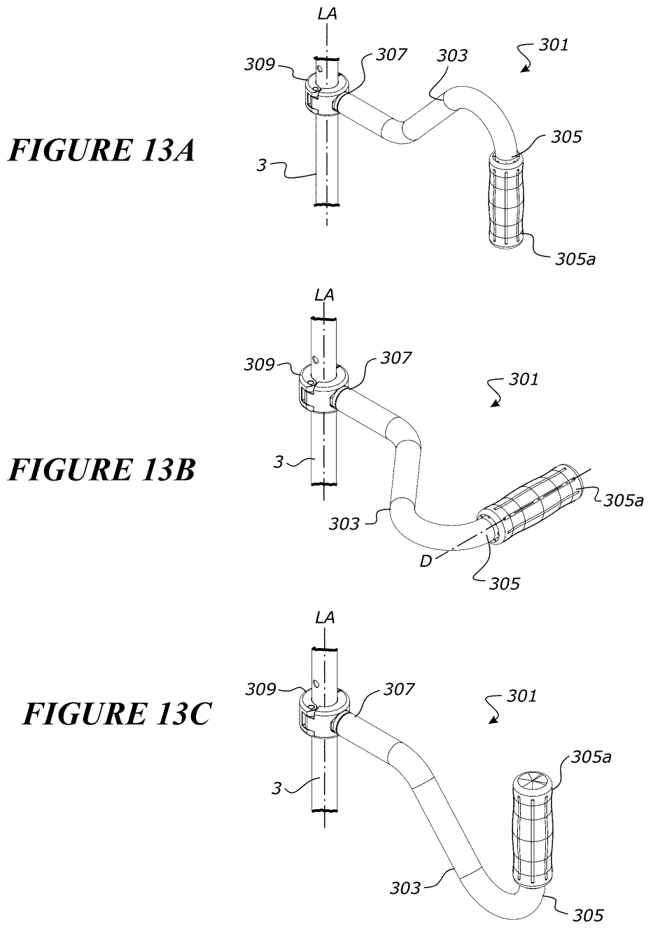

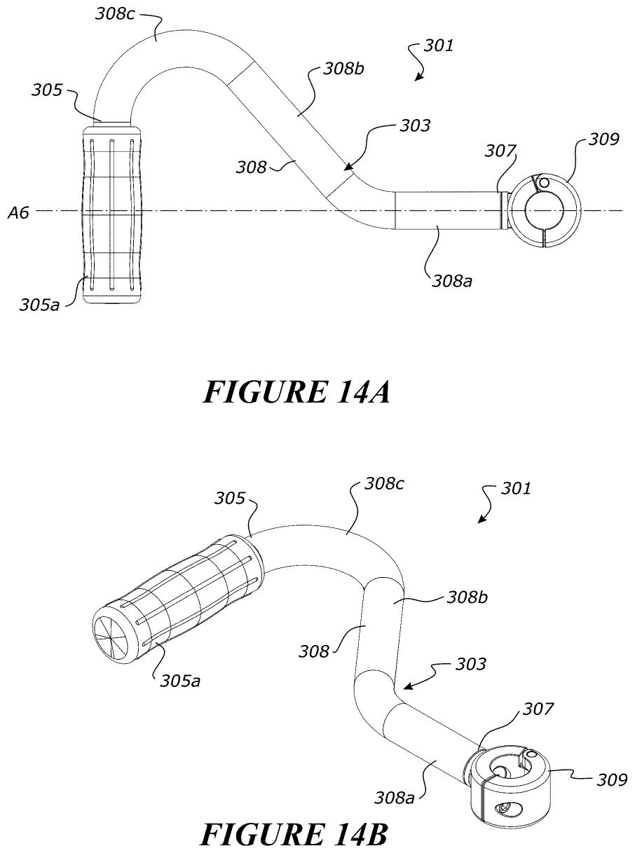

The midpoint of the grip 305a and the midpoint of the upright pole member 3 are collinear. This feature is shown by the axis A6 in FIG. 14A. This helps ensure the user has a neutral wrist action when contacting the grip 305a with a medium wrap grasp, or with full pronation. Therefore, this reduces the likelihood of ulnar or radial deviation during use due to torque created by a horizontal offset.

The medical pole handle 301 has a grip 305a comprising a sleeve on the grip end portion 305 of the handle body 303, to provide a comfortable and reliable gripping surface for a user. The grip 305a may comprise any suitable material such as an elastomeric material for example.

In an alternative configuration, the grip end portion 305 may not have an elastomeric sleeve. Instead, the grip end portion 305 may have a tactile gripping surface formed directly on the grip end portion 305. For example, the grip end portion 305 may comprise a knurled outer surface.

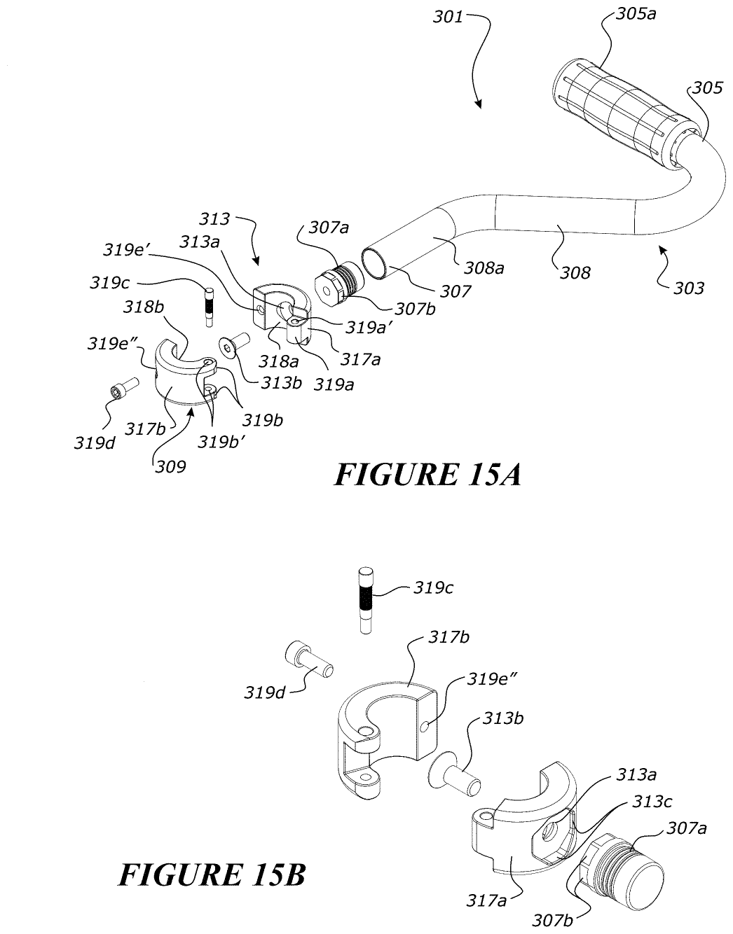

Referring to FIG. 15A, the mount 309 comprises a bracket or clasp that comprises a first clasp member or bracket part 317a and a second clasp member or bracket part 317b.

The first bracket part 317a is positioned proximal the mounting end portion 307 of the handle body 303. The second bracket part 317b is positioned distal the mounting end portion 307 of the handle body 303.

The first bracket part 317a and the second bracket part 317b are arranged to substantially surround the upright pole member 3 of the medical pole 1 from opposite sides, such that when the first bracket part 317a and the second bracket part 317b are closed around the upright pole member 3, the first bracket part 317a and the second bracket part 317b substantially surround the periphery of the upright pole member 3.

In the configuration shown, the first bracket part 317a and the second bracket part 317b have an arcuate shape with a substantially circular inner surface 318a, 318b to surround the circular periphery of a cylindrical upright pole member 3. However, the shapes of the inner surfaces 318a, 318b could differ from that shown, depending on the peripheral shape of the upright pole member 3.

The first bracket part 317a comprises part of the fastening mechanism 313 to engage the mounting end portion 307 of the handle body 303.

The fastening mechanism 313 comprises an aperture 313a through which a fastener 313b extends or is capable of extending, to engage with the mounting end portion 307 of the handle body 303.

As shown in FIG. 15A, an inner surface of the aperture 313a may be countersunk to receive the head of the fastener 313b, so that the head of the fastener 313b does not interfere with contact between the inner surface 318 and the upright pole member 3.

The mounting end portion 307 comprises an indexing member 307a that is non-rotatably mounted to the end of the mounting end portion 307. The indexing member 307a comprises an interstitial plug member which enables attachment of the mount 309 and handle body 303. The indexing member 307a may be one or more of a press fit, threaded connection, interference fit, welded connection, adhered connection, or integrally formed with the mounting end portion 307. Rotation of the indexing member 307a relative to the mounting end portion 307 is inhibited.

Alternative configurations could include other mechanisms by which to couple the mount 309 and handle body 303.

The indexing member 307a has an aperture that is complementary to the aperture 313a of the first bracket part 317a and that allows insertion of the fastener 313b. The fastener 313b can be any suitable type of fastener; for example, a screw or bolt. The aperture in the indexing member 307a is the same or substantially the same size as the aperture 313a. These apertures are aligned to allow for insertion of the fastener 313b.

The aperture 313a and the indexing member 307a of the mounting end portion 307 comprise complementary surface features 313c, 307b to fix the handle body 303 in the two or more orientations.

The surface features 313c are provided in a recess in the first bracket part 317a (FIG. 15B).

The complementary surface features 313c, 307b comprise angularly oriented surface features. In the form shown, the complementary surface features 313c, 307b comprise eight angularly oriented surface features in a generally octagonal configuration, which provides eight possible orientations for the handle body 303 relative to the mount 309. That is, this profile has rotational symmetry of order eight in the configuration shown.

This means there are eight discrete coupling positions for the mount 309 and handle body 303, allowing for adjustment of the handle position as shown in FIGS. 13A-13C and more. This profile could be shaped to allow a higher, or lower, number of coupling positions by defining a desired order of rotational symmetry.

In some configurations, there are at least four positions to align the grip end portion 305 horizontally or vertically. Accordingly, in an alternative configuration, the complementary surface features 313c, 307b may comprise four angularly oriented surface features in a generally square configuration, which provides four possible orientations.

In an alternative configuration, the complementary surface features 313c, 307b may comprise a different number of angularly oriented surface features, which provides a corresponding number of possible orientations.

The fastener 313b is concealed in the first bracket part 317a and the mounting end portion 307 so as to not be accessible or visible when the mount 309 is coupled to the medical pole 1.

Therefore, the medical pole handle 301 needs to be decoupled from the upright pole member 3 to change the orientation of the handle body 303. The concealment of the fastener 313b prevents unintended removal or change in the orientation of the handle body 303 during use.

The second bracket part 317b is hingedly coupled to the first bracket part 317a.

The mount 309 comprises a pivot arrangement to hingedly couple the second bracket part 317b to the first bracket part 317a. One of the first bracket part 317a and the second bracket part 317b comprises a projecting knuckle or tongue 319a that is received in a recess between two spaced-apart projecting knuckles or flanges 319b on the other of the first bracket part 317a and the second bracket part 317b. Alternatively, each of the bracket parts could have a single knuckle or flange. A pin 319c is received through apertures 319a′, 319b′ in the projecting tongue 319a and flanges 319b, to hingedly couple the second bracket part 317b to the first bracket part 317a.

When the second bracket part 317b is hinged into an open position relative to the first bracket part 317a, the mount 309 can be mounted to the upright pole member 3. The second bracket part 317b is then hinged into a closed position relative to the first bracket part 317a, and a fastener 319d is used to couple the free ends of the first bracket part 317a and the second bracket part 317b together, to engage the mount 309 to the upright pole member 3.

The second bracket part 317b and first bracket part 317a have complementary holes 319e″, 319e′ of the same or substantially the same size to receive the fastener 319d. The fastener could be any suitable type, such as a screw or bolt for example.

The hole 319e″ in the second bracket part 317b and the hole 319e′ in the first bracket part 317a are adapted to be aligned with each other so that the fastener 319d can engage with the holes when the mount 309 is closed.

When the fastener 319d is inserted through the holes 319e′, 319e″ and tightened, the first and second bracket parts 317a, 317b are coupled together to form a central aperture. The upright pole member 3 is adapted to be secured within the central aperture tightly enough so that the mount 309 does not slide up and down. Therefore, the diameter of the central aperture is substantially the same as or less than the diameter of the upright pole member 3 when the fastener 319d is tightened.

Also, the height of the medical pole handle 301 relative to the ground surface G can be adjusted by simply loosening the fastener 319d using a suitable tool and sliding the mount 309 along the longitudinal axis LA of the upright pole member 3. Once the mount 309 is slid to a desired height from the ground surface G, the fastener 319d may be tightened again to secure the mount 309 on the upright pole member 3.

Other configurations with a different number or arrangement of fasteners would be possible. This configuration mount 309 enables easy adjustment of handle height using only one fastener.

If the user wants to remove the medical pole handle 301 from the upright pole member 3 of the medical pole 1, they can remove the fastener 319d such that the second bracket part 317b can be hinged to the open position relative to the first bracket part 317a, and the medical pole handle 301 removed.

The first bracket part 317a and second bracket part 317b extend around a substantial portion of a perimeter of the upright pole member 3 of the medical pole 1 when the medical pole handle 301 is mounted to the medical pole 1 and the first bracket part 317a and the second bracket part 317b are closed.

In some configurations, the mount 309 and/or upright pole member 3 comprise complementary keying feature(s) or indexing feature(s) such that when the medical pole handle 301 is mounted to the upright pole member 3, it will be positioned over an extension leg 105.

In some configurations, the mount 309 and/or upright pole member 3 comprise complementary keying feature(s) or indexing feature(s) such that there is a discrete plurality of vertical mounting positions of the medical pole handle 301 along the upright pole member 3.

As shown, the orientation of the mount 309 does not change during adjustment. Only the orientation of the handle body 303 changes with respect to the mount 309, which means the handle body 303 can be adjusted between positions shown in FIGS. 13A to 13C. The ability to make adjustment of 90 degrees would allow a user to easily hold the grip end portion 305 in a vertical position or a horizontal position depending upon how the handle body 303 is adjusted. In an alternative configuration, the handle body may be adjusted at any angle with respect to the axis A6; e.g. the handle body 303 may be adjusted so that the grip end portion 305 is at 20 degrees, 40 degrees or any other angle with respect to the upright pole member 3 or ground surface G.

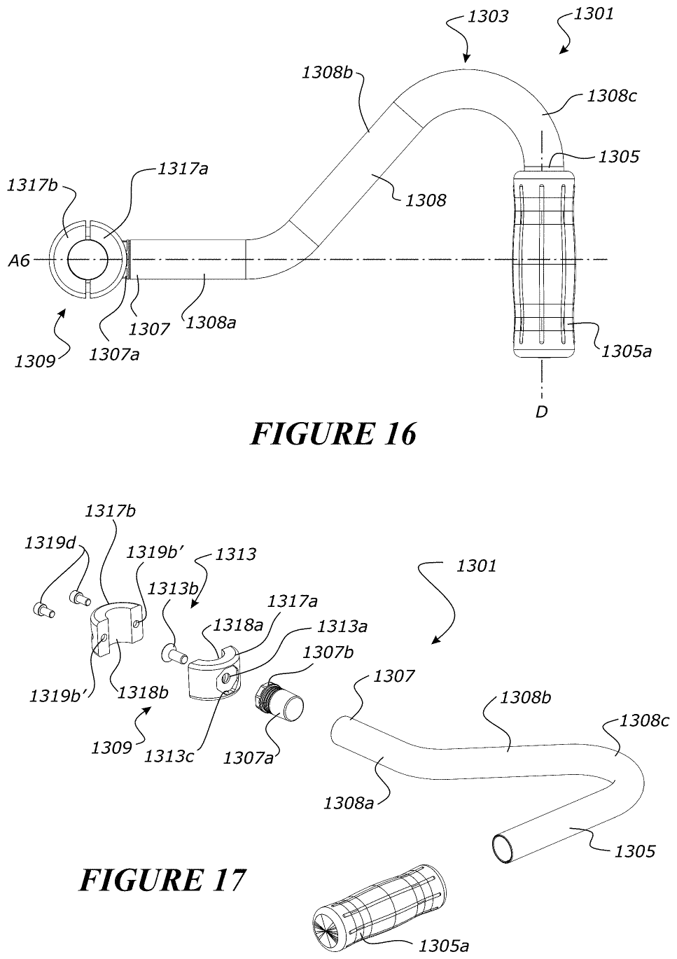

FIGS. 16 and 17 show an alternative configuration medical pole handle 1301. Unless described below, the features, functioning, and options are the same as for the medical pole handle 301, and like reference numerals indicate like parts with the addition of 1000.

The mount 1309 of this medical pole handle 1301 differs from the medical pole handle 301. Rather than the second bracket part 1317b being hingedly connected to the first bracket part 1317a, the second bracket part 1317b couples to the first bracket part 1317a in a linear movement. Two fasteners 1319d extend through apertures 1319b′ in the second bracket part 1317b and complementary apertures (not shown) in the first bracket part 1317a, to hold the mount in the engaged position around the upright pole member 3 of the medical pole 1.

The two apertures 1319b′ in the second bracket part 1317b and the complementary apertures in the first bracket part 1317a will be the same or substantially the same size, and are aligned with each other when the first bracket part 1317a and the second bracket part 1317b are positioned on opposite sides of the upright pole member 3.

Inserting the fasteners 1319d into the apertures and tightening them allows the first and second bracket parts 1317a, 1317b to be coupled together and form a central aperture, to hold the mount in the engaged position around the upright pole member 3 of the medical pole 1.

The fasteners 1319d can be removed by a user to remove the medical pole handle 1301 from the medical pole 1.

Other configurations with a different number or configuration of fasteners could be used.

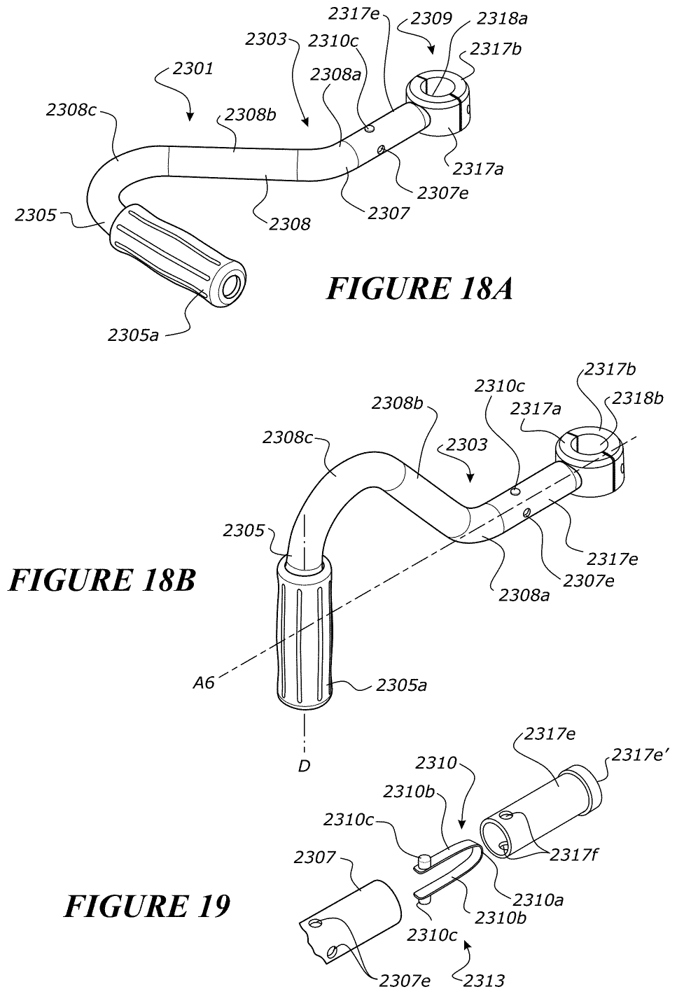

FIGS. 18A to 19 show an alternative configuration medical pole handle 2301. Unless described below, the features, functioning, and options are the same as for the medical pole handles 301 and 1301 and like reference numerals indicate like parts to medical pole handle 1301 with the addition of 1000.

In this configuration, the fastening mechanism 2313 comprises a spring clip arrangement. In the configuration shown, the spring clip arrangement comprises a double button spring clip arrangement.

The first bracket part 2317a has an extension 2317e extending from the first bracket part 2317a. The extension 2317e is coaxial with the axis A6.

An inner end 2317e′ of the extension 2317e is coupled to the first bracket part 2317a, either by being fastened or coupled to the first bracket part 2317a, or by being integrally formed with the first bracket part 2317a.

The extension 2317e comprise opposed concentric lateral or transverse apertures 2317f positioned adjacent an outer end of the extension 2317e.

The mounting end portion 2307 of the handle body 2303 comprises concentric lateral or transverse apertures 2307e that are spaced from the free end of the mounting end portion. In the configuration shown, the mounting end portion 2307 of the handle body 2303 comprises two pairs of opposed concentric apertures 2307e.

A spring clip 2310 is configured to couple the extension 2317e of the first bracket part 2317a with the mounting end portion 2307 of the handle body 2303.

The spring clip 2310 has a generally U-shaped configuration comprising a transverse base portion 2310a and two arms 2310b extending longitudinally out from the base portion. An outwardly oriented projection 2310c is located at or adjacent the free end of each arm 2310b.

The spring clip 2310 is made of a suitable resilient material, such that the projections 2310c are biased outwardly. For example, the spring clip 2310 could be made from spring steel or from a resilient polymeric material.

The spring clip 2310 is mounted in the extension 2317e with the base portion 2310a of the spring clip adjacent the first bracket part 2317a. The projections 2310c project outwardly through the opposed apertures 2317f in the extension 2317e. Depressing the projections 2310c allows for the insertion of the extension 2317e into the mounting end portion 2307 of the handle body. The projections 2310c are received in one pair of concentric apertures 2307e of the mounting end portion 2307 of the handle body 2303 to set the angular orientation of the medical pole handle 2301.

To adjust the angular orientation of the medical pole handle 2301, the projections 2310c are pushed laterally inwardly toward the axis A6 to clear the apertures 2307e, freeing the handle body 2303 for rotation relative to the extension 2317e.

Once the projections 2310c have freed to the apertures 2307e, the handle body 2303 can be rotated until the projections 2310c are aligned with apertures 2307e in the mounting end portion 2707. The resilience of the spring clip 2310 will cause the projections to bias outwardly through the apertures, inhibiting rotational movement of the handle body 2303 relative to the mount 2309 and thereby lock the orientation of the grip end portion 2305 relative to the upright pole member 3 of the medical pole 1.

Because the projections 2310c are biased outwardly, the projections 2310c will automatically engage with the apertures 2307e when they are aligned with the projections.

The handle body 2303 can be freely rotated relative to the mount 2309 when the projections 2310c are not engaged with the apertures 2307e. Rotation of the handle body 2303 relative to the mount 2309 is prevented when the projections 2310c are engaged with the apertures 2307e.

The number of apertures 2307e can be chosen depending on the number of desired angular orientations of the medical pole handle 2301 relative to the upright pole member 3 of the medical pole 1. For example, the mounting end portion 2707 may have two apertures 2307e to provide two opposed angular orientations of the handle 2310, may have four apertures 2307e to provide for opposed angular orientations of the handle 2310, or may have a different number of apertures 2307e and corresponding angular orientations.

This configuration enables the orientation of the handle body 2303 to be changed without decoupling the mount 2309 from the upright pole member 3.

Mechanisms other than a double button spring clip mechanism could enable a similar orientation change of telescopic tubes.

The medical pole handle 301, 1301, 2301 allows the medical pole 1 to be moved as required. The handle 301, 1301, 2301 allows manipulation of the medical pole from a distance which avoids any ergonomics issues with devices or accessories attached to the upright pole member 3 being in the way. The height of the handle 301, 1301, 2301 on the upright pole member 3 is independent of any devices or accessories. The height of various devices on the medical pole could have implications on patient or user safety.

The medical pole handle 301, 1301, 2301 allows easy manoeuvring and control of the medical pole 1. Further, the handle portion provided by the grip end portion 305, 1305, 2305 can be adjusted to different angular positions relative to the upright pole member 3 without requiring wieldy or complex tooling. The handle portion 301, 1301, 2301 is sleek and has low bulk, meaning that the medical pole handle is not only cosmetic but also does not block or accommodate large portions of the upright pole member 3 (which can carry a large number of accessories such as medications, equipment etc.) on which it is configured to be mounted.

The medical pole 1 may comprise an auxiliary grip portion 11 mounted vertically on the upright pole member 1. The grip auxiliary portion may comprise a grip member of a similar type described for the handle grip 3. The auxiliary grip portion 11 may be positioned above or below the medical pole handle 301, 1301, 2301. The auxiliary grip portion 11 works in conjunction with the medical pole handle 301, 1301, 2301 to enable two-handed gripping and manoeuvring of the medical pole 1.

FIGS. 20 to 26C show a medical apparatus identification system.

The identification system comprises a medical apparatus having a pole member, and two or more medical pole identification tags 401, 431, 461.

The medical apparatus identification system is shown and described in the context of a medical pole identification system for the medical pole 1 having the upright pole member 3. However, the medical apparatus identification system could be used for identification purposes with any suitable type of medical apparatus having a pole member. For example, the pole member may be provided on or as part of a medical apparatus that has a wheeled or non-wheeled base, and that has a pole member or pole-like member. The pole member or pole-like member may be an upright pole member or pole-like member, or could be on a different orientation relative to the ground surface G in use.

Therefore, depending on the application of the described identification system, the described medical pole could be a medical apparatus and the described upright pole member could be a pole member.

The pole member may have any suitable cross-sectional shape and dimension(s). In the example shown, the upright pole member 3 is a circular cylindrical cross-sectional shape having a diameter of about 1 inch (25.4 mm). The pole member could have a larger or smaller dimension, and/or a different cross-sectional shape such as an elliptical or oblong shape or a polygonal shape for example.

Single medical apparatus identification tags 401, 431, 461 may be used in the medical apparatus identification system, or the identification tags may be provided in set(s).

The identification tags 401, 431, 461 can be removably mounted to the upright pole member 3 of the medical pole 1 for identification purposes.

FIG. 20 shows three different configuration identification tags 401, 431, 461 engaged with the upright pole member 3 of the medical pole 1. The medical pole identification system may comprise a plurality of first configuration identification tags 401, a plurality of second configuration identification tags 431, and/or a plurality of third configuration identification tags 461.

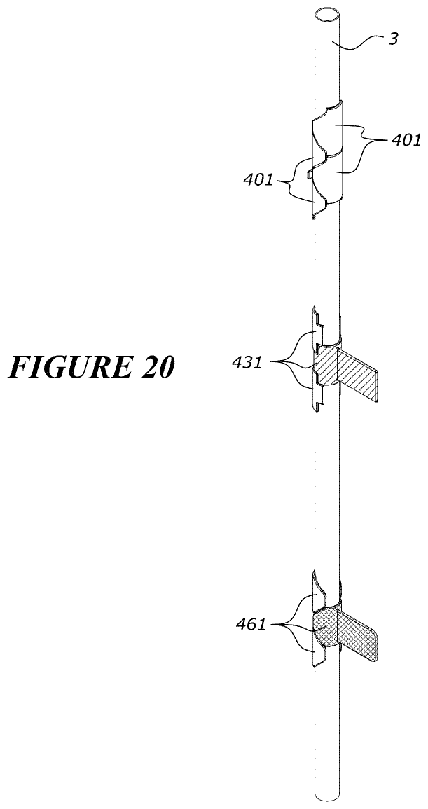

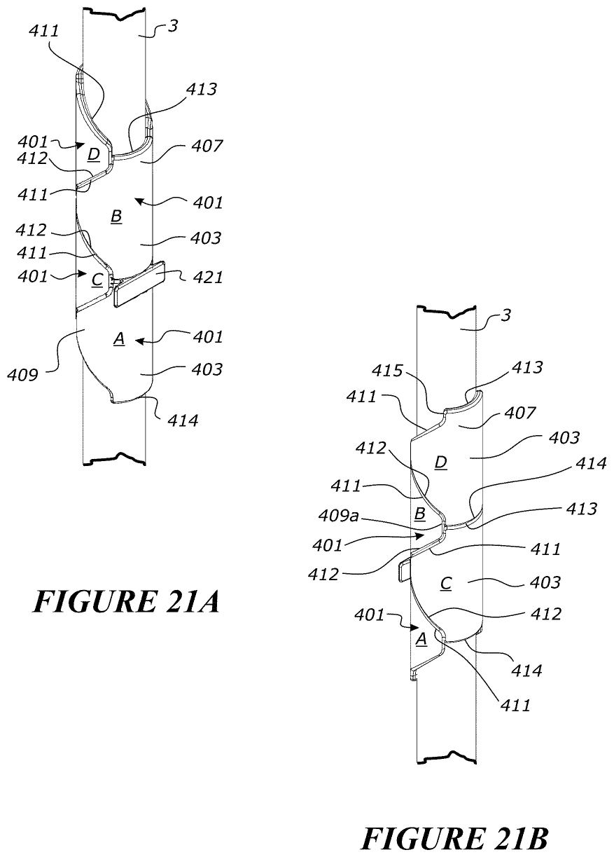

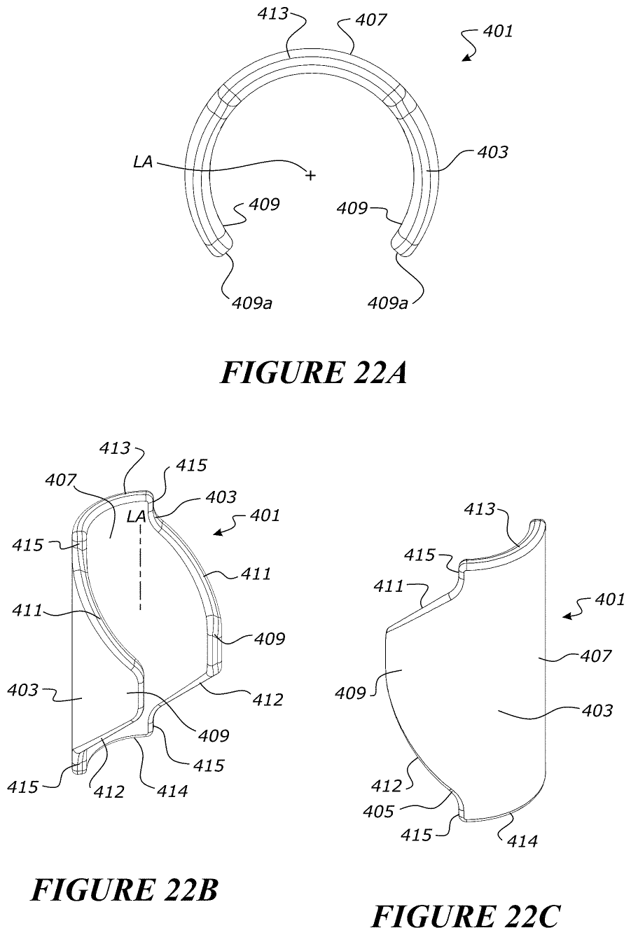

FIGS. 21A to 22C show the first configuration identification tags 401.

Each first configuration identification tag 401 comprises a clip portion 403. The clip portion 403 is arranged to engage with the medical pole 1.

In the configuration shown, the clip portion 403 is arranged to engage the upright pole member 3. An inner shape of the clip portion 403 is configured to engage with the peripheral surface of the upright pole member 3. In the form shown, the clip portion 403 has a substantially arcuate inner surface. The inner surface is a semi-circular arcuate shape to engage with the peripheral outer surface of the upright pole member 3. The clip portion 403 has a substantially C-shaped horizontal cross-section, transverse to its length.

The inner shape of the clip portion 403 could alternatively be a different shape to engage with a differently-shaped peripheral outer surface of the upright pole member 3.

The clip portion 403 comprises an elongate body 407 with opposing arms 409 extending from the elongate body 407. The clip portion 403 is configured to extend around more than a major part (i.e. more than 50%) of the periphery of the upright pole member 3 of the medical pole 1. In some configurations, the clip portion 403 is configured to extend around at least substantially an entirety of the periphery of the upright pole member 3 of the medical pole 1.

The elongate body 407 has a larger vertical height than the arms 409.

In the configuration shown, the arms 409 are substantially symmetrical in shape and size. Alternatively, the arms could differ from each other.

Each arm 409 extends from the body 407 at one end to another free end that is proximal to the corresponding free end of the other arm 409.

The clip portion 403 is configured to snap fit onto the upright pole member 3 of the medical pole 1. The identification tag 401 is made from a suitable resilient material. For example, the tag may be made from a resilient metal or polymeric material.

The identification tag 401 may, for example, be formed from: rigid material, resilient material, plastic(s), non-plastic rigid material(s), non-plastic resilient material(s), or combinations thereof.

The clip portion 403 is configured to engage the upright pole member 3 of the medical pole 1 in a direction that is substantially transverse to the longitudinal axis LA of the upright pole member 3 of the medical pole 1.

Due to the resilient nature of the identification tag 401, the arms 409 can be pulled apart by a user to enable the identification tag to engage with the upright pole member 3, and can then be released to snap into engagement with the upright pole member 3.

Alternatively, as shown in FIG. 22A, angled face surfaces 409a at free ends of the arms may be angled outwardly from inner edges of the angled face surfaces 409a to outer edges of the angled face surfaces 409a. As the clip portion 403 of the identification tag 401 is moved in the direction that is substantially transverse to the longitudinal axis LA of the upright pole member 3 of the medical pole 1, the upright pole member 3 will engage against the angled face surfaces 409a and cause the arms 409 to spread apart a sufficient distance to clear the widest portion at the centre of the upright pole member 3.

As the angled face surfaces 409a are moved beyond that widest portion of the upright pole member 3, the resilience of the arms 409 will draw the clip portion 403 into full engagement on the upright pole member 3, and the arms 409 will snap close around the upright pole member.

The clip portion 403 is configured to provide audible and/or haptic feedback to a user when the clip portion engages with the upright pole member 3 of the medical pole 1.

The arms 409 taper from one end to the other end.

Opposing edges 411, 412 of each arm 409 taper towards one another towards an axial centre of each arm from one end to the other end. In particular, the opposing edges 411, 412 taper towards each other from the connected ends of the arms to the free ends of the arms.

In some configurations, the free ends of the arms 409 that are distal the body 407 comprise a straight edge that is substantially parallel to a longitudinal axis LA of the identification tag (and of the upright pole member 403 in use). In alternative configurations, the arms comprise curved edges.

FIGS. 21A and 21B show a set of four first identification tags 401 (A, B, C, D) mounted to the upright pole member 3 of cylindrical cross section. All four identification tags 401 are of the same shape and size and each identification tag 401 removably mounts to the upright pole member 3 with snap fit arrangement. Using two or more of such identical identification tags 401 covers all of circumference of the pole without any visible gap, in the regions where the identification tags interact with each other. Therefore, the two or more identification tags 401 when mounted to the upright pole member 3 can be easily visible from all sides of the pole even from a distance. Additionally, this minimises the length of the upright pole member 3 that is covered by the identification tags 401. Also, having a set of identification tags 401 of the same size will mean that they can be easily mounted to the pole; i.e. the user need not worry about selecting one identification tags 401 over another when mounting multiple identification tags on the pole. The identification tags could be of different sizes and have different lengths.

The identification tags 401 are configured such that when they are engaged with the upright pole member 3 of the medical pole 1 with adjacent upper and lower edges 411, 412 of the identification tags 401 abutting each other, there is substantially no visible gap between the adjacent upper and lower edges 411, 412.

Each arm 409 comprises an edge 411, 412 that is complementary with an edge 411, 412 on the other arm 409, such that when two of the identification tags 401 are engaged with the upright pole member 3 of the medical pole 1, the complementary edges 411, 412 of one of the identification tags 401 abuts with the complementary edges 411, 412 of the other one of the identification tags 401, such that there is substantially no visible gap between the abutting complementary edges 411, 412.

As shown in FIGS. 21A and 21B, the identification tags 401 are configured such that when a plurality of the identification tags are engaged with the upright pole member 3 of the medical pole 1, the identification tags 401 cooperate to extend around substantially an entirety of the periphery of the medical pole.

The adjacent upper and lower side edges 411, 412 of the adjacent identification tags 401 abut each other, upper and lower transverse body edges 413, 414 of the adjacent identification tags 401 abut each other, and upper and lower extension portions 415 that extend from the arms 409 to the upper and lower body edges 413, 414 engage with the free end face surfaces 409a of the adjacent identification tags 401.

The identification tags 401 are configured such that when they are engaged with the medical pole, they cooperate to enable a user to view a portion of each identification tag 401 from any angle around the medical pole 1, when the user views the identification tags at eye level.

In some configurations, at least one of the identification tags comprises a protrusion.

In the configuration shown in FIGS. 21A and 21B, one of the identification tags 401 comprises a protrusion 421 providing an optional gripping portion, prising lip or removal grip. The protrusion may be integrally formed with the clip portion 403 of the identification tags 401. In an alternative configuration, the gripping portion may not be formed integrally with the clip portion 403 of the identification tags 401.

Having a protrusion 421 is advantageous as a user may use his/her fingers to grip the protrusion 421 to easily pull the identification tag 411 out from the pole member or to easily push the identification tag to snap fit to the pole member. The protrusion 421 need not be located at the position as shown and can be located at any external portion of the clip portion 403. This protrusion 421 could take alternate forms, such as a hoop or ring for example, and the user is not limited to a particular set of motions or tools by which to couple/decouple the identification tag 401 from the pole member 3.

In some configurations, the protrusion 421 is substantially planar.

In some configurations, the protrusion 421 comprises a writing surface.

In the configuration shown, the protrusion 421 extends transversely to the longitudinal axis LA of the identification tag 401, tangentially to the body 407, and horizontally in use. In an alternative configuration, the protrusion 421 could extend at a different angle to the clip portion 403, such as radially from the outer surface of the clip portion 403 for example.

At least a portion of each identification tag 401 comprises an identification feature. The identification may identify the medical pole 1 when in use.

In some configurations, the identification feature comprises at least one identification colour. The identification colour may be for identifying the medical pole. The colour and clip portion 403 allows a user to identify the medical pole by the colour of the identification tag 401 from various angles.

In some configurations, the identification tags comprise different identification colours.

In some configurations, each identification tag has a single identification colour. In some configurations, each identification tag comprises more than one identification colour.

As an example, in FIGS. 21A and 21B, identification tags A and B may be yellow, identification tag C may be green, and identification tag D may be red. That colour combination may indicate that the medical pole 1 on which the identification tags are mounted belongs to a department of the hospital, e.g. emergency department. Similarly, if the identification tags 401 that are mounted to the pole are all red that may indicate something different, e.g. the pole belongs to a patient receiving respiratory therapy treatment carried by the pole. In the same way, a colour combination of green, yellow, orange and pink may indicate that the pole on which the identification tags are mounted belongs to patient that is being supervised by a medical practitioner. Similarly, a combination of colours that appears as green, yellow, red, blue respectively when viewed from top to bottom of the medical pole may indicate something different that the combination that appears yellow, red, blue and green when respectively when viewed from top to bottom.

Similarly, the number of identification tags may also be used for identification. For example, three identification tags mounted to the upright pole member 3 may mean the medical pole is carrying breathing assistance apparatus whereas seven identification tags may mean that the medical pole is carrying surgical equipment.

These examples are non-limiting examples of using the identification tags for identification.

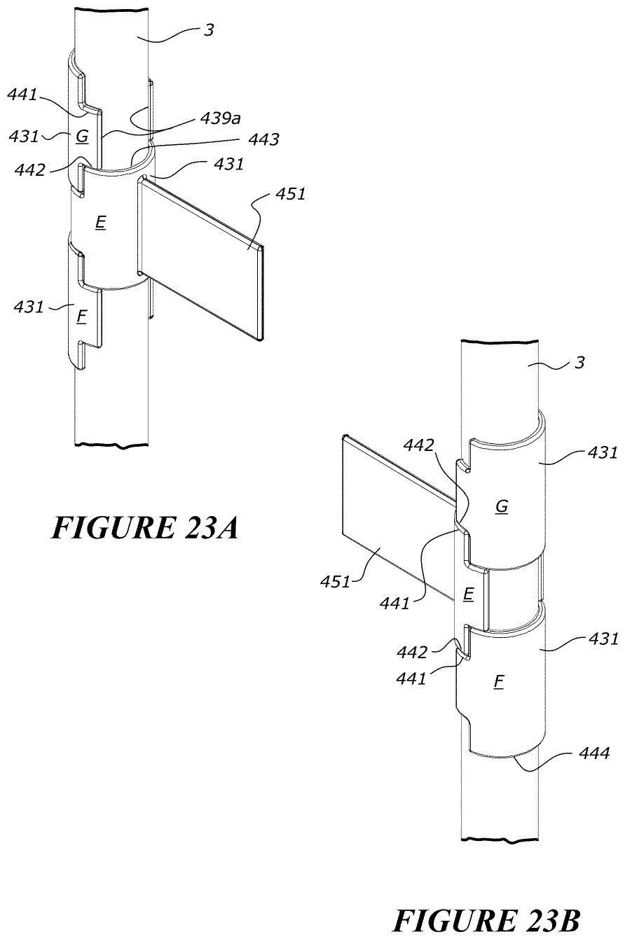

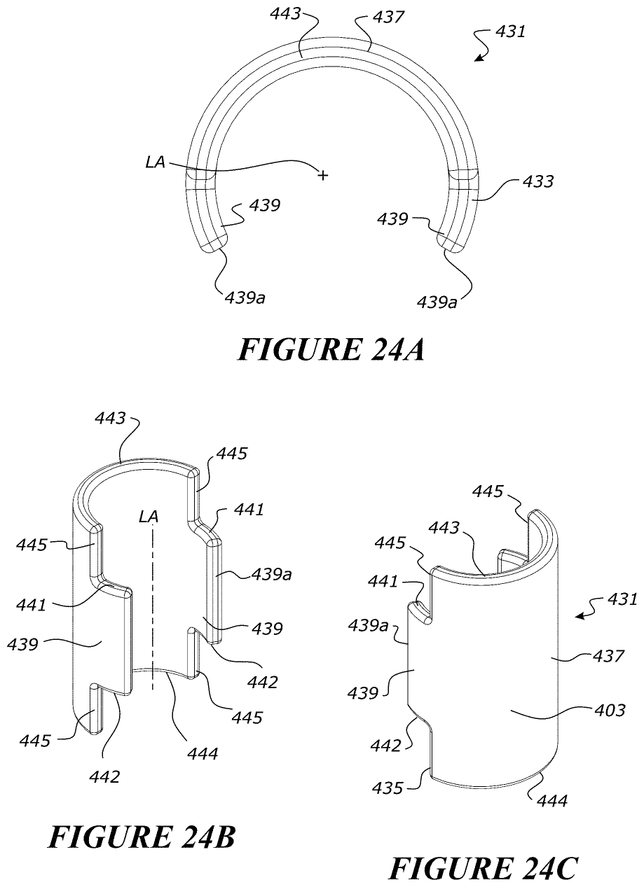

FIGS. 23A-24C show a second configuration medical pole identification tag 431. Unless described below, the features, functioning, and options are the same as for identification tag 401, and like reference numerals indicate like parts with the addition of 30.

In this configuration, an edge 441, 442, 445 of each arm 439 that is configured to be positioned adjacent an edge of an arm 439 of an adjacent identification tag 431, comprises at least one step.

FIGS. 23A and 23B show a set of three identification tags 431 (identification tag E, identification tag F, identification tag G) of FIGS. 24A-24C that are mounted to the upright pole member 3.

All identification tags 431 may be of the same shape and size and each identification tag removably mounts to the upright pole member 3 with a snap fit arrangement.

A protrusion 451 extends radially from the clip portion 433. The protrusion 451 forms a gripping portion and is of a substantial size to allow labels/stickers comprising information to be place on it. The protrusion 451 may comprise a writing surface so that a user may write any information (e.g. patient's name) on this protrusion 451 using a marker pen or any other suitable writing implement. Any colour, colour combination and/or number of identification tags may be used for identification purposes similar as described for identification tags 401.

As seen in FIG. 23B, when a plurality of identification tags 431 are mounted to the upright pole member 3 in stacked arrangement, gaps are present exposing a few portions of the upright pole member 3. In other words, unlike the identification tags 401, the identification tags 431 cannot fully cover the circumference of the upright pole member 3 in the region where the identification tags 431 are mounted to the upright pole member 3. The sides of the identification tags do, however, have edges 441, 442 that abut each other so there are no gaps between those edges.

It can be appreciated that due to the gaps, not all identification tags 431 may be visible from all sides of the medical pole, especially from distance.

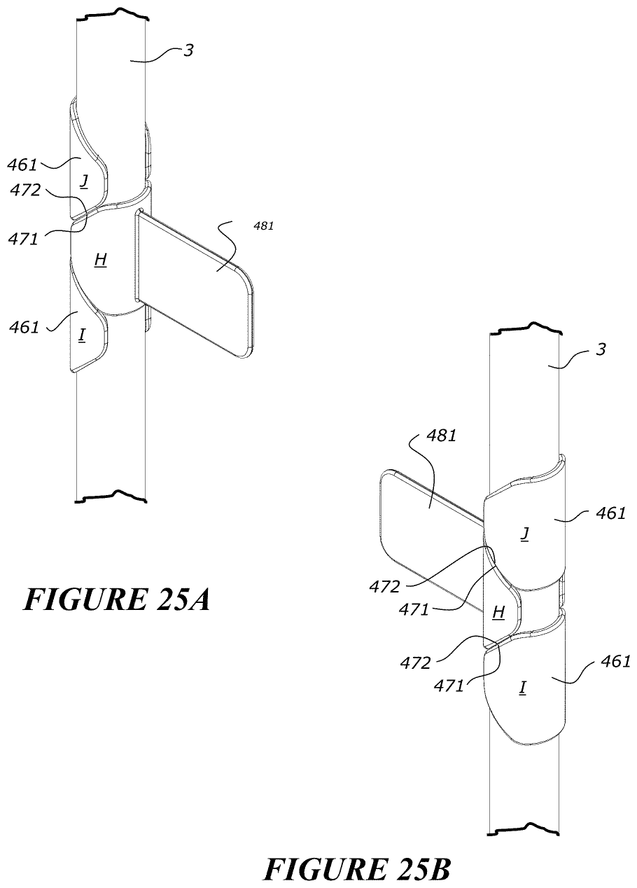

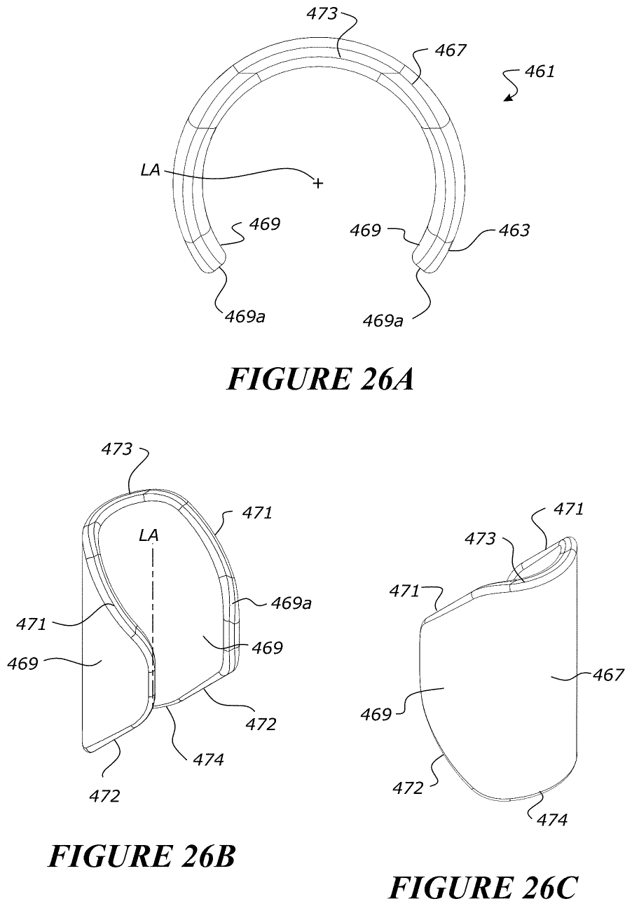

FIGS. 25A-26C show a third configuration medical pole identification tag 461. Unless described below, the features, functioning, and options are the same as for identification tag 401, and like reference numerals indicate like parts with the addition of 60.

In this configuration, there is a smooth transition from the body 467 to the arms 469. Therefore, the portions of the arms 469 proximal the body 467 have substantially the same height as the body 467.

FIGS. 25A and 25B show a set of three identification tags 461 (identification tag H, identification tag I, and identification tag 3) mounted to the upright pole member of cylindrical cross section. Any colour, colour combination and/or number of identification tags 461 may be used for identification purposes similar as described above.

As seen in FIG. 25B, when a plurality of identification tags 461 are mounted to the upright pole member 3 in stacked arrangement, gaps are present exposing few portions of the pole member. In other words, unlike the identification tags 401, the identification tags 461 cannot fully cover the circumference of the pole member 3 in the region where the identification tags 461 are mounted to the pole. The sides of the identification tags do, however, have edges 471, 472 that abut each other so there are no gaps between those edges.

It can be appreciated that due to the gaps, not all identification tags 461 may be visible from all sides of the medical pole, especially from distance.

The identification tags 401, 431, 461 could have shapes that differ from those shown.

Depending on the size of a hospital or patient care facility, there can be large number of medical poles or other medical apparatuses. In such situations, it can be challenging to determine several things such as but not limited to:
details of patient receiving treatment from medications and/or equipment carried by the medical pole or other medical apparatus
medical practitioner treating/supervising the patient associated with the medical pole or other medical apparatus
medications and, equipment and/or accessories carried by the medical pole or other medical apparatus
ward or department the medical pole or other medical apparatus belongs to
conditions for which treatment is being received by a patient associated with a medical pole or other medical apparatus.

The identification tag(s) 401, 431, and/or 461 provides one or more of the following advantages:
can be easily seen from a distance
well secured on the medical pole or other medical apparatus
conveniently and easily usable
can provide a surface suitable for the receiving labels
self-contained and need no further construction by the user prior to use
can cover all, or substantially all, of the circumference of the pole member on which the clip or set of clips are mounted
capable of being produced in multiple colours
reusable
flexible and durable in construction.

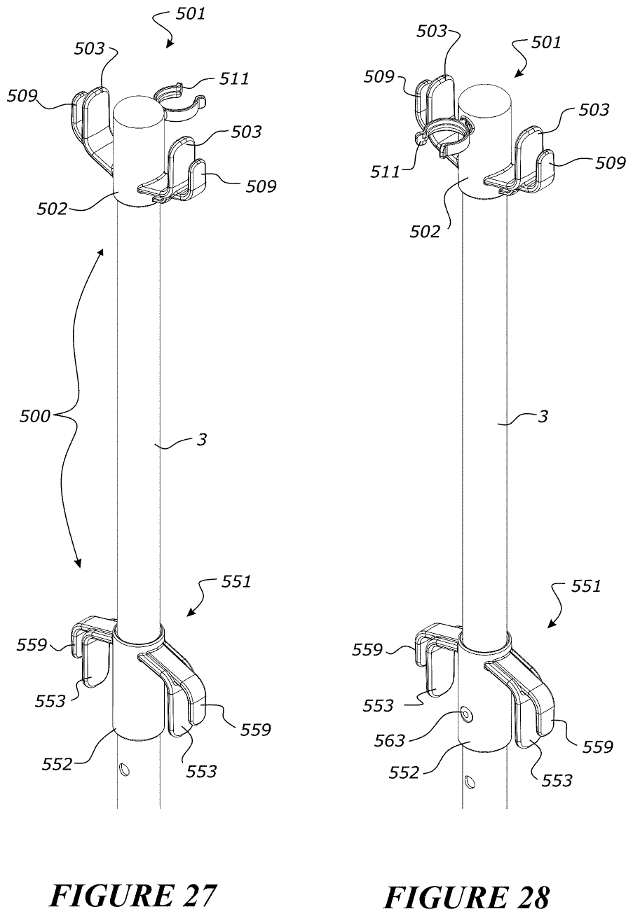

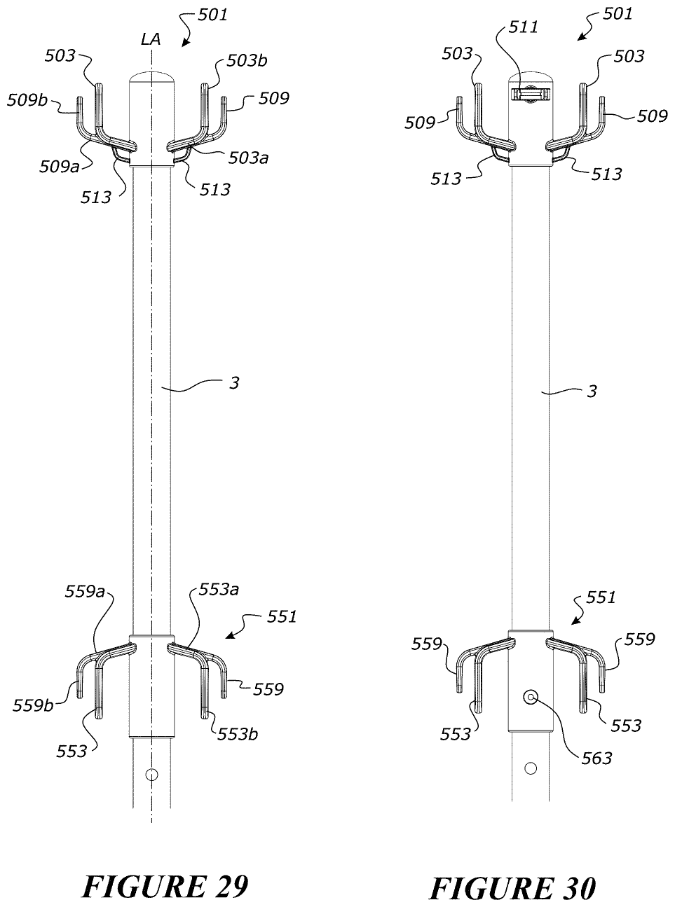

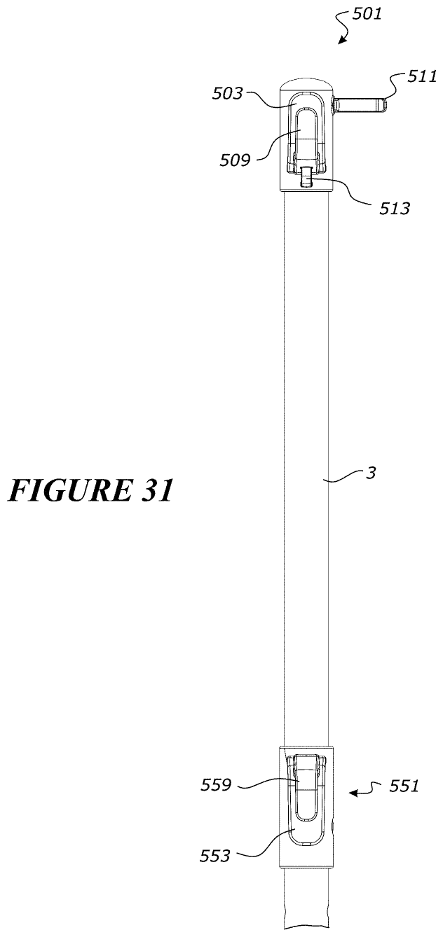

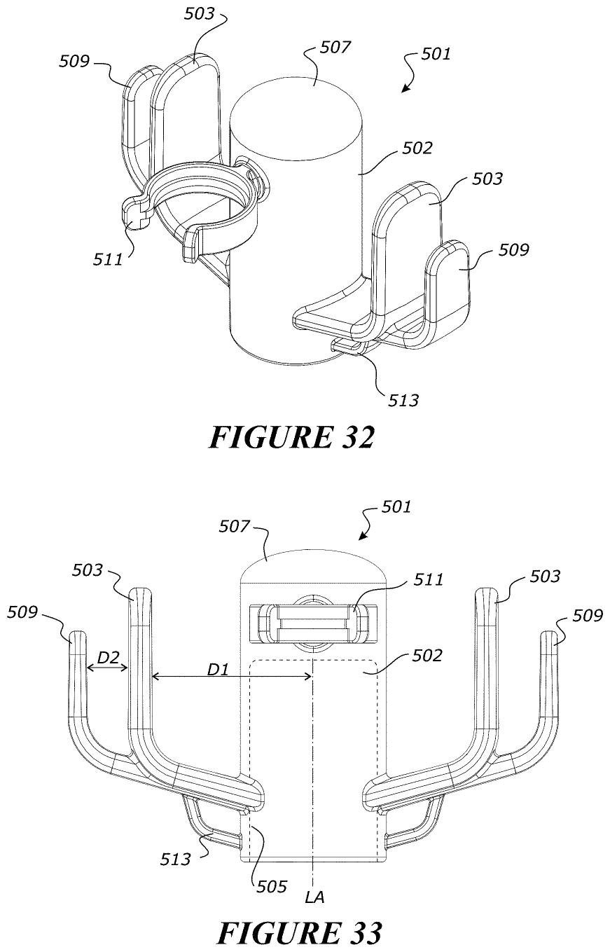

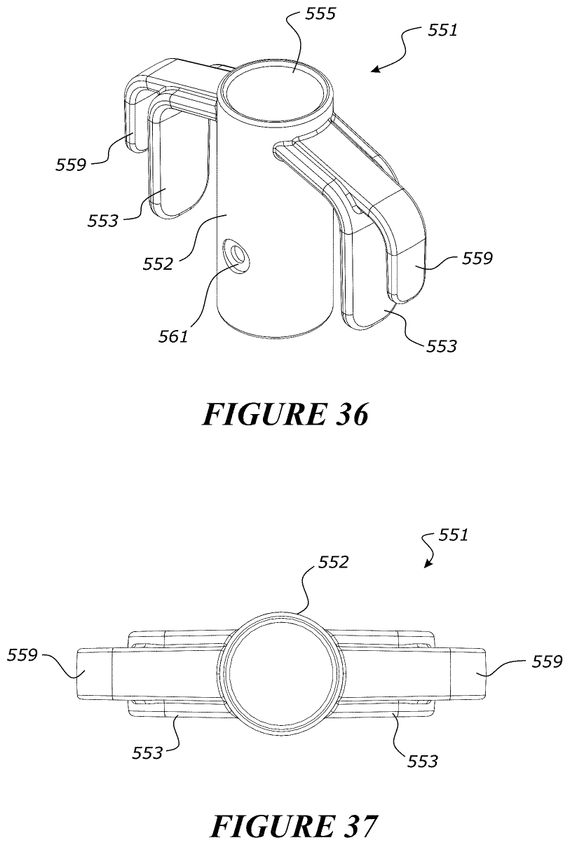

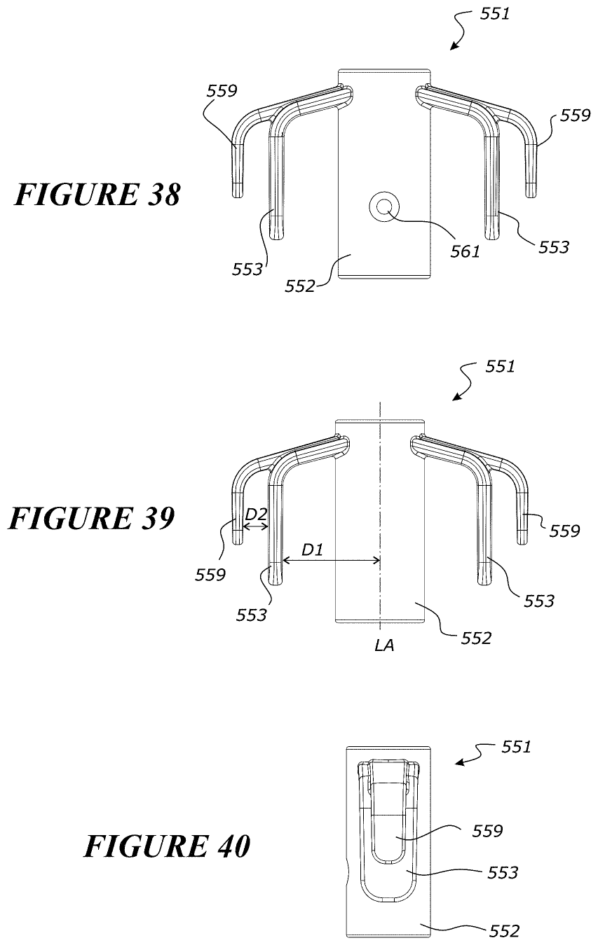

With reference to FIGS. 27 to 42, the medical pole 1 comprises a retaining assembly 500 to enable management of one or more peripheral components such as conduit(s), cable(s), breathing circuit element(s), a liquid bag for a humidifier, and/or swing tag(s). The retaining assembly 500 may be used to wind, wrap, and/or otherwise secure the component(s) to the medical pole 1.

The retaining assembly 500 comprises a first retaining arrangement 501 and a second retaining arrangement 551 on the upright pole member 3.

The first retaining arrangement 501 is an upper retaining arrangement of the retaining assembly 500, and comprises an upwardly extending hook 503 that extends outwardly and upwardly from a body 502 of the first retaining arrangement.

The upwardly extending hook 503 comprises a laterally extending base portion 503a and an extension portion 503b. In the configuration shown, the laterally extending base portion 503a is oriented upwardly at a non-horizontal angle, and the extension portion 503b is substantially parallel to the longitudinal axis LA of the upright pole member 3. These configurations could vary.

The second retaining arrangement 551 is a lower retaining arrangement of the retaining assembly, and comprises a downwardly extending hook 553 that extends outwardly and downwardly from a body 552 of the second retaining arrangement.

The downwardly extending hook 553 comprises a laterally extending base portion 553a and an extension portion 553b. In the configuration shown, the laterally extending base portion 553a is oriented downwardly at a non-horizontal angle, and the extension portion 553b is substantially parallel to the longitudinal axis LA of the upright pole member 3. These configurations could vary.

The upper retaining arrangement 501 is spaced apart along the upright pole member 3 from the lower retaining arrangement 551. The upper and lower hooks 503, 553 are spaced apart along the upright pole member 3 of the medical pole 1.

The spacing between the upper and lower hooks 503, 553 is sufficient for a peripheral component to be wound around the opposed hooks. In some configurations, the spacing between the upper and lower hooks 503, 553 is about 400 mm+/−100 mm; however, any suitable spacing may be provided depending on the length of peripheral component to be supported.

The retaining arrangements 501, 551 comprise a channel or recess 505, 555 in the respective body 502, 552 to receive the upright pole member 3 of the medical pole 1. In the configuration shown, the bodies 502, 552 completely surround the periphery of the upright pole member 3. In an alternative configuration, the bodies 502, 552 may be open to one or more sides to partly surround the periphery of the upright pole member 3.

The channel or recess 505, 555 may allow the retaining arrangement 501, 551 to move axially along the upright pole member 3.

The first retaining arrangement 501 comprises an end cap 507. The end cap 507 covers the upper end of the upright pole member 3 in use, to prevent debris entering the upright pole member from above.

The first retaining arrangement 501 is coupled to an upper end of the upright pole member 3.

The second retaining arrangement 551 is coupled to an intermediate portion of the upright pole member 3 along its length.

The hooks 503, 553 are arranged substantially along the same plane, on a same side of the upright pole member 3. That enables an elongate member to be wound around the hooks 503, 553 without needing to wrap the elongate member around the pole member 3.

The hooks 503, 553 extend outwardly from the respective body 502, 552, so as to be arranged about or outwardly of the respective channel 505, 555.

The first retaining arrangement 501 comprises a securing mechanism.

The body 502 of the first retaining arrangement 501 may be a hollow plastic moulded object, with an integral end cap 507 and internal ribs. In this configuration, the internal ribs may act as the securing mechanism for securing the first retaining arrangement 501 to the upright pole member 3. The body 502 can be press fit onto the top of the upright pole member 3. It will be appreciated that other materials and means of securing the upper retaining arrangement 501 to the upright pole member 3 could be utilised. For example, the first retaining arrangement may be secured to the pole mechanism by one or more fasteners and/or adhesive.

Each retaining arrangement 501, 551 may comprise a single hook. Alternatively, each retaining arrangement 501, 551 may comprise at least two hooks 503, 553 arranged on opposite sides of each retaining arrangement. In the configuration shown, the first retaining arrangement 501 comprises two of the upwardly extending hooks 503 and the second retaining arrangement comprises two of the downwardly extending hooks 553. The at least two hooks are symmetrical about the longitudinal axis LA.

In another configuration, each retaining arrangement 501, 551 may comprise three, four, or more hooks 503, 553.

Each retaining arrangement 501, 551 comprises a primary hook 503, 553. The primary hook is a relatively large hook. The primary hook may be suitable for winding a conduit, tube, or any other longitudinal flexible component for example.

The primary hook 503, 553 is positioned a first, relatively large, distance D1 from a center of the respective retaining arrangement. The distance D1 defines a relatively large channel in the primary hook 503, 553.

In one exemplary configuration, distance D1 may be about 35 mm and the size of the large channel between the body 502 and the hook 503 may be about 19 mm at the upper end tapering to 18 mm at the lower end. Such a configuration may impart a small compressive retaining force onto a respiratory conduit. Any suitable distance and size may be provided depending on the intended use of the retaining arrangement.

Each retaining arrangement 501, 551 comprises a secondary hook 509, 559. The secondary hook 509, 559 is a relatively small hook. The secondary hook may be suitable for winding power lines and/or hanging liquid bag(s) for example.

The auxiliary hook 509, 559 is connected to and extends outwardly from the respective primary hook 503, 553.

The auxiliary hook 509, 559 is positioned a second distance D2 from the respective primary hook 503, 553. The second distance D2 is less than the first distance D1. The second distance D2 defines a relatively small channel in the secondary hook 509, 559.

In one exemplary configuration, distance D2 may be about 10 mm, but may be several mm larger or smaller, depending on the intended use of the auxiliary hook 509.

In the form shown, the secondary hooks 509, 559 are shorter and transversely narrower than the primary hooks 503, 553. Other than that, the physical features of the secondary hooks 509, 559 may be the same as the respective primary hooks 503, 553, and like reference numbers indicate like parts with the addition of 6.

The sizes, spacings, and numbers of different primary hooks 503, 553 and secondary hooks 509, 559 could vary.

When a plurality of primary hooks 503, 553 are present, there may be a corresponding plurality, or fewer, secondary hooks 509, 559.

The free end of each hook 503, 509, 553, 559 may be rounded and/or tapered to assist with inserting accessories onto the hooks.

The first retaining arrangement 501 comprises a conduit clip element 511. The conduit clip element can be used to secure a conduit during respiratory therapy for example.

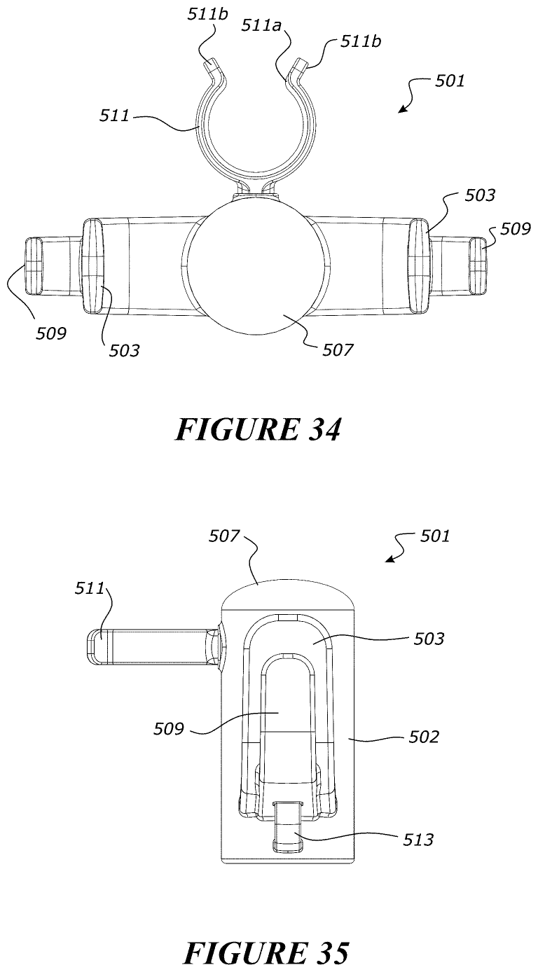

The conduit clip element 511 extends from a side of the body 502 and has a generally C-shaped configuration. A mouth 511a (FIG. 34) of the conduit clip element 511 is provided between outwardly directed members 511b configured such that the conduit can be inserted laterally into the conduit clip element with a snap fit, similar to the engagement of the identification tags 401, 431, 461 onto the upright pole member 3.

The first retaining arrangement 501 comprises a loop element 513 that defines a loop arranged below the upwardly extending hook 503. When a plurality of upwardly extending hooks are present, there may be a single loop element 513 or a plurality of loop elements 513. The loop element(s) can be used to hold a swing tag or another ancillary or user interface element.

The cylindrical body 552 of the second retaining arrangement 551 may be a hollow moulded plastic object with an inner diameter larger than the upright pole member 3 to be fit to the pole member 3 in a concentric manner. The second retaining arrangement 553 comprises a securing mechanism, to secure the second retaining arrangement 553 to the upright pole member 503. The securing mechanism may releasably couple the second retaining arrangement to the pole member.

The second retaining arrangement comprises an aperture 561 through which a fastener 563 such as a screw or rivet can be driven to secure the second retaining arrangement to the upright pole member 3. The upright pole member 3 may comprise one or more apertures for receiving the fastener 563. When a plurality of apertures is provided in the upright pole member, the height of the second retaining arrangement on the upright pole member 3 could be selected or adjusted. Other types of attachment would also be possible, such as a grub screw or alternative fastener arrangement.

The first and second retaining arrangements 501, 551 may be symmetric about a vertical plane extending through a longitudinal axis, or may be asymmetric in an alternative configuration. Alternative configurations could include fewer, or a greater number of, channels, hooks, and loops. The channels, and the hook elements which define them, may be substantially similar (i.e. the distances D1 and D2 could be substantially the same).

Instead of an end cap 507 and a press fit attachment to the top of the upright pole member 3, the first retaining arrangement 501 may use a securing mechanism that is substantially similar to the second retaining arrangement 551. Therefore, the configuration of the first retaining arrangement 501 could be substantially similar to that of the second retaining arrangement 551, with the hook orientation inverted.

Rather than being secured to the upright pole member 3 of the medical pole 1, one or both of the first and second retaining arrangements 501, 551 could be integrally formed with the upright pole member 3.

In an alternative configuration, the first and second retaining arrangements 501, 551 could be integrally formed with each other and secured to the upright pole member 3 as one item.

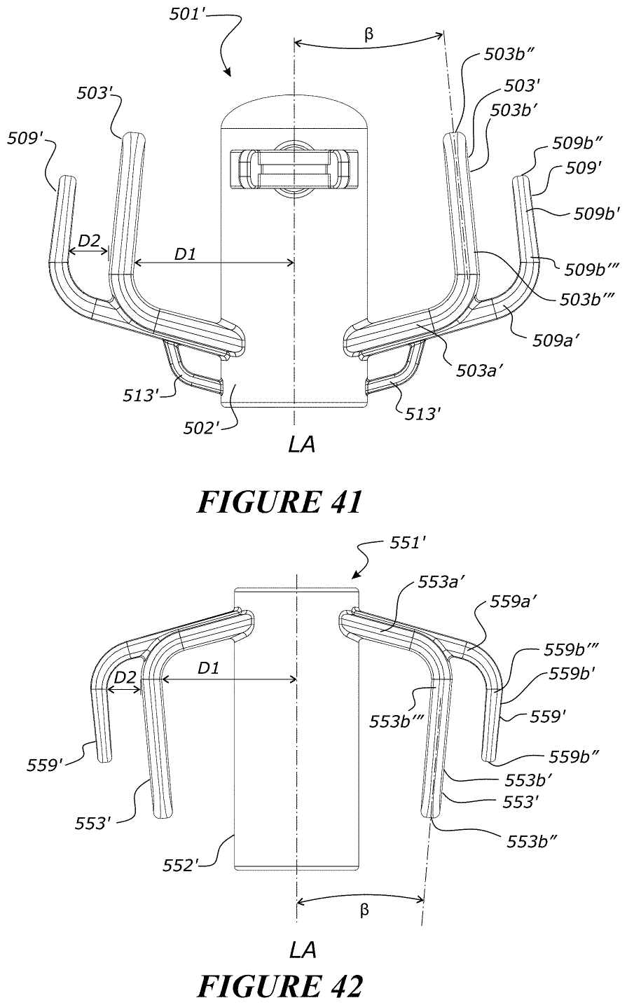

FIG. 41 shows an alternative configuration first retaining arrangement 501′. Unless described below, the features, functioning, and options for the alternative configuration first retaining arrangement 501′ are the same as for the first retaining arrangement 501. Like numbers indicate like parts with the addition of a prime (′).

The upwardly extending hook(s) 503′ of the alternative configuration first retaining arrangement 501′ comprise(s) a laterally extending base portion 503a′ and an extension portion 503b′. The extension portion 503b′ is angled inwardly toward the centre of the alternative configuration first retaining arrangement 501′ to provide a relatively narrow opening adjacent a free end 503b″ of the extension portion.

The extension portion 503b′ has the free end 503b″ and a fixed portion 503b″. The fixed portion 503b′″ connects the extension portion 503b′ to the base portion 503a′ of the hook. The free end 503b″ of the extension portion of the hook is closer to the centre of the retaining arrangement 501′ than the fixed portion 503b′″. The opening adjacent the free end 503b″ of the extension portion is narrower than a distance between the fixed portion 503b′″ and the body 502′.

The extension portion 503b′ is angled toward the longitudinal axis LA of the upright pole member, and thereby toward the centre of the retaining arrangement 501′, at angle β. The angle β may be more than 0 degrees and up to about 5 degrees. For example, the angle β may be more than 0 degrees, about 1 degree, about 2 degrees, about 3 degrees, about 4 degrees, or about 5 degrees.

The configuration with the inwardly angled extension portion 503b′ provides a relatively narrow opening between the free end 503b″ of the extension portion and the body 502′. The relatively narrow opening assists with retaining the peripheral component, e.g. a conduit, on the hook 503′. This is beneficial especially when the medical pole is being moved.

The hook(s) 503′ may be primary hook(s). The retaining arrangement 501′ may comprise secondary hook(s) 509′ as described above in relation to the first retaining arrangement 501.

The extension portion 509b′ of the secondary hook 509′ may be substantially parallel to the extension portion 503b′ of the primary hook 503′. Therefore, the extension portion 509b′ may be angled toward the longitudinal axis LA of the upright pole member at angle β.

Alternatively, the extension portion 509b′ of the secondary hook 509′ may be angled inwardly toward the extension portion 503b′ of the primary hook 503′. In that configuration, the free end 509b″ of the secondary hook 509′ is closer to the primary hook 503′ than the fixed portion 509b′″ of the secondary hook 509′.

The extension portion 509b′ of the secondary hook 509′ may be angled inwardly toward the extension portion 503b′ of the primary hook 503′ at an angle of more than 0 degrees and up to about 5 degrees. For example, the angle may be more than 0 degrees, about 1 degree, about 2 degrees, about 3 degrees, about 4 degrees, or about 5 degrees.

The same configuration but with downwardly extending hooks with angled extension portions could be used in an alternative configuration second retaining arrangement 551′ as shown in FIG. 42. The features, functioning, and options for the alternative configuration second retaining arrangement 501′ are the same as described above for the alternative configuration first retaining arrangement 501′, but the hooks are inverted. Like numbers indicate like parts to FIG. 41 with the addition of 50.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Features from any of the described embodiments may be combined with each other and/or an apparatus may comprise one, more, or all of the features of the above described embodiments. Moreover, not all of the features, aspects and advantages are necessarily required to practice the present disclosure. Accordingly, the scope of the present disclosure is intended to be defined only by the claims that follow.

The various configurations described are exemplary configurations only. Any one or more features from any of the configurations may be used in combination with any one or more features from any of the other configurations.

For example, the components and features are described with reference to an upright pole member 3 of a medical pole 1. Alternatively, the components and features could be used with a pole member having a different configuration. For example, the pole member could be operatively connected to the upright pole member 3, and could be parallel to the upright pole member, perpendicular to the pole member, or angled so as to be non-perpendicular and non-parallel relative to the upright pole member 3. The pole member could be integrally formed with or coupled to the upright pole member 3.

As another example, the upright pole member 3 is described as having a substantially circular peripheral shape, with the components of the medical pole 1 shaped accordingly. The pole member could have any suitable peripheral shape, including but not limited to a polygonal shape, a symmetric shape, or an asymmetric shape, with the components of the medical pole 1 shaped accordingly.

Reference to any prior art in this specification is not, and should not be taken as, an acknowledgement or any form of suggestion that the prior art forms part of the common general knowledge in the field of endeavour in any country in the world.

Where reference is used herein to directional terms such as ‘up’, ‘down’, ‘forward’, ‘rearward’, ‘horizontal’, ‘vertical’ etc, those terms refer to when the apparatus is in a typical in-use position and/or with reference to particular orientations shown in the figures, and are used to show and/or describe relative directions or orientations.

1. A medical pole comprising: a movable base supporting a pole member, the movable base comprising a frame and two or more wheels, the two or more wheels comprising a first wheel and a second wheel, the first wheel comprising a first wheel first mode in which the first wheel is swivelable about a first wheel respective upright axis relative to the frame and is rollable about a first wheel respective transverse axis, and the first wheel comprising a first wheel second mode in which the first wheel is rollable about the first wheel respective transverse axis but is non-swivelable about the first wheel respective upright axis, the first wheel being configurable between the first wheel first mode and the first wheel second mode.

2. The medical pole of claim 1, wherein the second wheel comprises a second wheel first mode in which the second wheel is swivelable about a second wheel respective upright axis relative to the frame and is rollable about a second wheel respective transverse axis, and a second wheel second mode in which the second wheel is non-rollable about the second wheel respective transverse axis and is non-swivelable about the second wheel respective upright axis, the second wheel being configurable between the second wheel first mode and the second wheel second mode.

3. The medical pole of claim 2, wherein the first wheel and the second wheel are arranged substantially opposite each other with the pole member located substantially between the first wheel and the second wheel.

4. The medical pole of claim 2, wherein the first wheel comprises a locking mechanism capable of selecting the first wheel first mode or the first wheel second mode and/or the second wheel comprises a locking mechanism capable of selecting the first second wheel first mode or the second wheel second mode.

5. The medical pole of claim 4, wherein the first wheel has a fixed orientation about the first wheel respective upright axis relative to the frame in the first wheel second mode.

6. The medical pole of claim 5 wherein the first wheel further comprises one or more fixed orientation positions about the first wheel respective upright axis.

7. The medical pole of claim 6 wherein the first wheel comprises four fixed orientation positions about the first wheel respective upright axis.

8. The medical pole of claim 7, wherein the four fixed orientation positions are substantially orthogonal to each other.

9. The medical pole of claim 6, wherein the first wheel is able to move into the one or more fixed orientation positions relative to the frame if the first wheel is not in any of the one or more fixed orientation positions relative to the frame when the locking mechanism of the first wheel is activated.

10. The medical pole of claim 6, wherein at least one of the fixed orientation positions is parallel to an intended movement direction of the medical pole.

11. (canceled)

12. The medical pole of claim 4, wherein an orientation of the second wheel about the second wheel respective upright axis relative to the frame is fixed upon activation of the locking mechanism of the second wheel.

13.-17. (canceled)

18. The medical pole of claim 1, wherein the frame comprises at least one extension leg that extends perpendicularly relative to the pole member with a respective one of the first wheel and the second wheel being arranged along the extension leg.

19.-21. (canceled)

22. The medical pole of claim 1 further comprising a handle extending from the pole member.

23. The medical pole of claim 22, wherein the handle extends from the pole member in a direction that is substantially perpendicular to an axis extending through the first wheel and the second wheel, and the handle extending from the pole member in a direction that is substantially parallel to and over an extension leg.

24. The medical pole of claim 22, wherein the handle extends from the pole member in a direction away from the first wheel and the handle extending from the pole member in a transverse or orthogonal direction away from the first wheel or the handle extending from the pole member in a direction that is opposite to the first wheel.

25. (canceled)

26. The medical pole of claim 1, wherein the first wheel comprises a locking mechanism capable of selecting the first wheel first mode or the first wheel second mode, and/or the second wheel comprises a locking mechanism capable of selecting a second wheel first mode or a second wheel second mode, and/or a third wheel comprises a locking mechanism capable of selecting a third wheel first mode or a third wheel second mode, the locking mechanism comprising a depressible lever.

27. The medical pole of claim 26, wherein the locking mechanism comprises a tooth/teeth and slot(s) arrangement, the depressible lever being configured to cause engagement of the tooth/teeth and slot(s) to lock swiveling of the wheel.

28. The medical pole of claim 27, wherein the first wheel comprises fewer tooth/teeth and slot(s) compared to the second wheel and/or the third wheel.

29. The medical pole of claim 26, wherein the locking mechanism comprises a brake member that is actuable by the depressible lever to engage a surface of the first wheel and/or the second wheel and/or the third wheel that contacts a ground surface when in use.

30.-55. (canceled)
